(19) United States
(12) Patent Application Publication
(10) Pub. No.: US 2026/0174363 A1
(43) Pub. Date: Jun. 25, 2026

(54) SELECTIVE COATING OF SENSOR METHOD AND DEVICES

(71) Applicant: Dexcom, Inc., San Diego, CA (US)

(72) Inventors: Jiong Zou, San Diego, CA (US); Shanger Wang, San Diego, CA (US); Ted T. Lee, San Diego, CA (US); Kristoffer Roy C. Ravana, San Diego, CA (US); Florante A. Pineda, San Diego, CA (US); Mahender Nath Avula, San Diego, CA (US); Saral Kalanadhabhatla, San Diego, CA (US); Joshua Ray Windmiller, San Diego, CA (US); Andre Filipe Camargo Silva, San Diego, CA (US); Becky L. Clark, San Diego, CA (US); Anna C. Harley-Trochimczyk, San Diego, CA (US); William D. Davis-Betanzos, San Diego, CA (US); Samuel Charles Rogers, San Diego, CA (US); Jay Philip Warren, San Diego, CA (US); Sean Akio Collignon, San Diego, CA (US); Max Walter Akita, San Diego, CA (US); Jonathan Kyle Harris, San Diego, CA (US)

(73) Assignee: Dexcom, Inc., San Diego, CA (US)

(21) Appl. No.: 19/343,954

(22) Filed: Sep. 29, 2025

Related U.S. Application Data
(60) Provisional application No. 63/738,498, filed on Dec. 23, 2024.

(51) Int. Cl. A61B 5/1473; A61B 5/00; A61B 5/145

(52) U.S. Cl. CPC A61B 5/14735; A61B 5/14532; A61B 5/6851; A61B 2562/125

(57) ABSTRACT

The present disclosure relates generally to apparatuses and methods of selectively coating along a length of an elongated body with a coating. In some examples, the elongated body is an insertable analyte sensor portion and the coating comprises one or more anti-inflammatory compounds or tissue response modifiers. One or more additional layers are also disclosed that modulate a release rate or an amount of the one or more anti-inflammatory compounds or tissue response modifiers after implantation in interstitial fluid.

### CROSS-REFERENCE TO RELATED APPLICATIONS

This application claims the benefit of U.S. Provisional Application No. 63/738,498 filed on Dec. 23, 2024, the entirety of which is incorporated herein by reference.

### TECHNICAL FIELD

The present disclosure relates generally to apparatuses and methods of selectively coating along a length of an elongated body with a coating. In some examples, the elongated body is an insertable analyte sensor portion and the coating comprises one or more anti-inflammatory compounds or tissue response modifiers.

### BACKGROUND

One of the most heavily investigated analyte sensing devices is the implantable glucose device for detecting glucose levels in hosts with diabetes. Despite the increasing number of individuals diagnosed with diabetes and recent advances in the field of implantable glucose monitoring devices, currently used devices are unable to provide data safely and reliably for certain periods of time due to local tissue responses. By way of example, there are two commonly used types of subcutaneously implantable glucose sensing devices. These types include those that are implanted transcutaneously and those that are wholly implanted.

### SUMMARY

In examples, an apparatus is provided, the apparatus comprising a reservoir comprising a coating solution, an orifice fluidically coupled to the reservoir, the orifice configured to present a meniscus comprising the coating solution, a holder for reversibly introducing at least one substrate to be at least partially coated in proximity to the orifice.

In aspects, the orifice has a rectangular shape, a rounded rectangle shape, a stadium shape, a squircle shape, a frustum shape, or a slit shape. In aspects, alone or in combination with any one of the previous aspects, the orifice has a width and a length greater than the width. In aspects, alone or in combination with any one of the previous aspects, the width to length ratio is less than 0.1.

In aspects, alone or in combination with any one of the previous aspects, the orifice is continuous along the length. In aspects, alone or in combination with any one of the previous aspects, the orifice is discontinuous along the length.

In aspects, alone or in combination with any one of the previous aspects, the reservoir comprises a pump. In aspects, alone or in combination with any one of the previous aspects, the pump is a low shear pump. In aspects, alone or in combination with any one of the previous aspects, the pump is a peristaltic pump.

In aspects, alone or in combination with any one of the previous aspects, the pump is configured to continuously present the coating solution to the orifice. In aspects, alone or in combination with any one of the previous aspects, the pump is configured to semi-continuously present the coating solution to the orifice.

In aspects, alone or in combination with any one of the previous aspects, the orifice is configured to present the coating solution to the orifice in a pulsatile manner. In aspects, alone or in combination with any one of the previous aspects, the orifice is horizontally arranged.

In aspects, alone or in combination with any one of the previous aspects, the orifice is horizontally arranged such that the meniscus is presented upwardly from the orifice. In aspects, alone or in combination with any one of the previous aspects, the orifice is configured to dispense the coating solution against a gravitational force. In aspects, alone or in combination with any one of the previous aspects, the orifice is horizontally arranged such that the meniscus is presented downwardly from the orifice.

In aspects, alone or in combination with any one of the previous aspects, the coating solution comprises at least one polymer or polymerizable monomer. In aspects, alone or in combination with any one of the previous aspects, the coating solution comprises at least one polymer or polymerizable monomer in combination with at least one drug.

In aspects, alone or in combination with any one of the previous aspects, the coating solution comprises a polymer chain having polyurethane and/or polyurea segments. In aspects, alone or in combination with any one of the previous aspects, the coating solution comprises a polymer chain having both hydrophilic and hydrophobic regions. In aspects, alone or in combination with any one of the previous aspects, the coating solution comprises a polymer chain having one or more zwitterionic compounds.

In aspects, alone or in combination with any one of the previous aspects, the coating solution comprises a polymer with a heterocyclic group. In aspects, alone or in combination with any one of the previous aspects, the coating solution comprises a polymer chain having poly(1-vinyl imidazole), poly(4-vinyl pyridine), poly(2-vinyl pyridine), acrylonitrile, acrylamide, and/or copolymers quaternized forms thereof. In aspects, alone or in combination with any one of the previous aspects, the one or more membranes comprises a copolymer including styrene.

In aspects, alone or in combination with any one of the previous aspects, the coating solution comprises a conductive polymer. In aspects, alone or in combination with any one of the previous aspects, the coating solution comprises a doped conductive polymer.

In aspects, alone or in combination with any one of the previous aspects, the coating solution comprises an anti-inflammatory compound or a tissue response modifier. In aspects, alone or in combination with any one of the previous aspects, the coating solution comprises a derivative form of dexamethasone, dexamethasone acetate, or a combination of a derivative form of dexamethasone or dexamethasone acetate with dexamethasone.

In aspects, alone or in combination with any one of the previous aspects, the coating solution comprises a nitric oxide (NO) releasing molecule, polymer, or oligomer.

In aspects, alone or in combination with any one of the previous aspects, wherein each of the at least one substrate comprises a longitudinal axis. In aspects, alone or in combination with any one of the previous aspects, the at least one substrate is an analyte sensor. In aspects, alone or in combination with any one of the previous aspects, the at least one substrate is a glucose sensor.

In aspects, alone or in combination with any one of the previous aspects, the at least one substrate is an elongated body or is a plurality of elongated bodies. In aspects, alone or in combination with any one of the previous aspects, the elongated body is a wire, an insulated wire, an insulated wire with skived portions, planar, or combinations thereof.

In aspects, alone or in combination with any one of the previous aspects, the holder is configured to present each of the at least one substrates to the orifice. In aspects, alone or in combination with any one of the previous aspects, the holder is configured to horizontally present the longitudinal axis of the at least one substrate to the orifice.

In aspects, alone or in combination with any one of the previous aspects, the holder is configured to present the longitudinal axis of the at least one substrate orthogonally to the longitudinal axis of the orifice. In aspects, alone or in combination with any one of the previous aspects, the holder is further configured to vertically adjust each of the at least one substrates. In aspects, alone or in combination with any one of the previous aspects, the holder is computer controlled.

In other examples, a method of selectively coating a portion of an elongated body is provided, the method comprising providing a meniscus, the meniscus, the meniscus having a length and a width and comprising a coating solution, contacting the elongated body with the meniscus, the elongated body along having a longitudinal length, forming a coated portion on the portion of the elongated body, and removing the elongated body from the meniscus thereby resulting in the coated portion and an uncoated portion of the elongated body.

In aspects, the uncoated portion is adjacent the coated portion along the longitudinal length. In aspects, alone or in combination with any one of the previous aspects, the coated portion is adjacent opposing sides of the uncoated portion along the longitudinal length.

In aspects, alone or in combination with any one of the previous aspects, the meniscus has a rectangular shape, a rounded rectangle shape, a stadium shape, a squircle shape, a frustum shape, or a slit shape.

In aspects, alone or in combination with any one of the previous aspects, the meniscus has a width and a length greater than the width, the width of the meniscus corresponds to the portion along the longitudinal length of the elongated body. In aspects, alone or in combination with any one of the previous aspects, the width to length ratio is less than 0.1.

In aspects, alone or in combination with any one of the previous aspects, the meniscus is continuous along the length. In aspects, alone or in combination with any one of the previous aspects, the meniscus is discontinuous along the length.

In aspects, alone or in combination with any one of the previous aspects, the meniscus is configured to continuously present the coating solution. In aspects, alone or in combination with any one of the previous aspects, the meniscus is configured to semi-continuously present the coating solution.

In aspects, alone or in combination with any one of the previous aspects, the meniscus is configured to present the coating solution in a pulsatile manner. In aspects, alone or in combination with any one of the previous aspects, the elongated body is presented horizontally and orthogonally relative to the meniscus.

In aspects, alone or in combination with any one of the previous aspects, the coating solution comprises at least one polymer or polymerizable monomer. In aspects, alone or in combination with any one of the previous aspects, the coating solution comprises at least one polymer or polymerizable monomer in combination with at least one drug.

In aspects, alone or in combination with any one of the previous aspects, the coating solution comprises a polymer chain having polyurethane and/or polyurea segments. In aspects, alone or in combination with any one of the previous aspects, the coating solution comprises a polymer chain having both hydrophilic and hydrophobic regions.

In aspects, alone or in combination with any one of the previous aspects, the coating solution comprises a polymer chain having one or more zwitterionic compounds.

In aspects, alone or in combination with any one of the previous aspects, the coating solution comprises a polymer with a heterocyclic group. In aspects, alone or in combination with any one of the previous aspects, the coating solution comprises a polymer chain having poly(l-vinyl imidazole), poly(4-vinyl pyridine), poly(2-vinyl pyridine), acrylonitrile, acrylamide, and/or copolymers quaternized forms thereof. In aspects, alone or in combination with any one of the previous aspects, the coating solution comprises a copolymer including styrene.

In aspects, alone or in combination with any one of the previous aspects, the coating solution comprises a conductive polymer. In aspects, alone or in combination with any one of the previous aspects, the coating solution comprises a doped conductive polymer.

In aspects, alone or in combination with any one of the previous aspects, the coating solution comprises an anti-inflammatory compound or tissue response modifier. In aspects, alone or in combination with any one of the previous aspects, the coating solution comprises a derivative form of dexamethasone, dexamethasone acetate, or a combination of a derivative form of dexamethasone or dexamethasone acetate with dexamethasone.

In aspects, alone or in combination with any one of the previous aspects, the elongated body comprises at least one analyte sensor. In aspects, alone or in combination with any one of the previous aspects, the elongated body comprises at least one glucose sensor.

In aspects, alone or in combination with any one of the previous aspects, the at least one analyte sensor has a sensing portion configured to generate a signal associated with a concentration of an analyte.

In aspects, alone or in combination with any one of the previous aspects, the coating of the coating solution on the portion of the elongated body is spatially separated from the analyte portion. In aspects, alone or in combination with any one of the previous aspects, the elongated body is a wire, an insulated wire, an insulated wire with skived portions, planar, or combinations thereof.

In aspects, alone or in combination with any one of the previous aspects, the elongated body comprises at least one electrode.

In other examples, the device for measurement of an analyte concentration is provided, the device comprising an analyte sensing portion configured to generate a signal associated with a concentration of an analyte, and a bioactive agent releasing portion configured to release a bioactive agent. The bioactive agent-releasing portion and the analyte sensing portion are spatially separated along a longitudinal axis of the wire substrate or the planar substrate, or the bioactive agent-releasing portion and the analyte sensing portion are present on separate wire substrates or separate planar substrates.

In aspects, the analyte sensing portion is present on the planar substrate. In aspects, alone or in combination with any one of the previous aspects, the analyte sensing portion is present on the wire substrate. In aspects, alone or in combination with any one of the previous aspects, the bioactive agent releasing portion is present on the planar substrate. In aspects, alone or in combination with any one of the previous aspects, the bioactive agent releasing portion is present on the wire substrate.

In aspects, alone or in combination with any one of the previous aspects, the bioactive agent-releasing portion and the analyte sensing portion are spatially separated. In aspects, alone or in combination with any one of the previous aspects, the bioactive agent-releasing portion and the analyte sensing portion are spatially separated along a longitudinal axis of the wire substrate or the planar substrate.

In aspects, alone or in combination with any one of the previous aspects, the analyte sensing portion comprises a WE, a RE, and/or CE configured to generate a signal associated with the analyte.

In aspects, alone or in combination with any one of the previous aspects, the bioactive agent-releasing portion comprises at least one bioactive agent-releasing electrode. In aspects, alone or in combination with any one of the previous aspects, the bioactive agent-releasing electrode is distal from the WE or RE. In aspects, alone or in combination with any one of the previous aspects, the WE and the bioactive agent-releasing electrode share the CE or the RE.

In aspects, alone or in combination with any one of the previous aspects, the analyte sensing portion comprises a first WE configured to generate a signal associated with a first analyte. In aspects, alone or in combination with any one of the previous aspects, the analyte sensing portion comprises a second WE configured to generate a signal associated with a second analyte, the second analyte being chemically different from the first analyte.

In aspects, alone or in combination with any one of the previous aspects, the bioactive agent-releasing electrode is positioned most distal relative to any other WE electrode.

In aspects, alone or in combination with any one of the previous aspects, the device further comprises an electrically conductive membrane in proximity to the bioactive agent-releasing electrode, the electrically conductive membrane comprising at least one bioactive agent, the at least one bioactive agent configured to be released from the electrically conductive membrane to modify tissue response of a subject.

In aspects, alone or in combination with any one of the previous aspects, the electrically conductive membrane comprises at least one electrically conductive polymer. In aspects, alone or in combination with any one of the previous aspects, the at least one electrically conductive polymer is doped.

In aspects, alone or in combination with any one of the previous aspects, the signal is potentiometric, coulometric, or amperometric.

In other examples, a device for measurement of an analyte concentration is provided, the device comprising an analyte sensing portion configured to generate a signal associated with a concentration of an analyte, a bioactive agent releasing portion adjacent the analyte sensing portion, the bioactive agent releasing portion configured to release a bioactive agent upon implantation in interstitial fluid, and a photon-activated layer adjacent the bioactive agent releasing portion, where the photon-activated layer comprises one or photo-sensitive agents.

In other examples, a method of controlling drug release from an implantable portion an analyte sensor is provided, the method comprising providing an analyte sensor comprising an implantable portion configured to generate a signal associated with a concentration of an analyte; a bioactive agent releasing portion adjacent the analyte sensing portion, the bioactive agent releasing portion configured to release a bioactive agent upon implantation in interstitial fluid; a photon-activated layer adjacent the bioactive agent releasing portion, the photon-activated layer comprises one or more photo-sensitive agents and reduces a release rate or amount released of the bioactive agent. The photon-activated layer is configured to modify the release rate or the amount of the bioactive agent from the bioactive agent releasing portion upon exposure to an amount of photon energy.

### BRIEF DESCRIPTION OF THE DRAWINGS

FIG. 1A is a perspective view schematic illustrating layers of an in vivo portion of a continuous analyte sensor, as shown and described herein.

FIG. 1B is a side-sectional view schematic illustrating an in vivo portion of a continuous analyte sensor, as shown and described herein.

FIG. 1C is a side-sectional view schematic illustrating an in vivo portion of a continuous analyte sensor, as shown and described herein.

FIG. 1D is a side-sectional view schematic illustrating an in vivo portion of a continuous analyte sensor, as shown and described herein.

FIG. 1E is a perspective view schematic illustrating an in vivo portion of a continuous analyte sensor as disclosed and described herein.

FIG. 1F is a perspective view schematic illustrating an in vivo portion of a continuous multi-electrode, multi-analyte sensor.

FIG. 1G is an expanded perspective schematic of section 1F of FIG. 1F showing the distal portion of the sensor example.

FIGS. 2A-2C are cross-sectional views of a sensor illustrating various embodiments of a membrane system coated as shown and described herein.

FIG. 3A is a perspective view schematic illustrating a meniscus coating apparatus as disclosed and described herein.

FIG. 3B is a schematic illustrating selectively positioned meniscus coating a plurality of elongated bodies using the meniscus coating apparatus as disclosed and described herein.

FIG. 3C is a schematic illustrating selectively positioned meniscus coated elongated bodies as disclosed and described herein.

FIG. 4A is a side view schematic illustrating an elongated body being selectively positioned coated using a meniscus coating apparatus as disclosed and described herein.

FIG. 4B is an expanded view of section 4B of FIG. 4A.

FIG. 5A is a schematic illustrating selective positional coating a plurality of elongated bodies using a vertical meniscus coating apparatus as disclosed and described herein.

FIG. 5B is an expanded view of section 5B of FIG. 5A.

FIG. 6A is a schematic illustrating tip coating a plurality of elongated bodies using a wall meniscus coating apparatus as disclosed and described herein.

FIG. 6B is a schematic illustrating a plurality of tip coated elongated bodies using a wall meniscus coating apparatus as disclosed and described herein.

FIG. 7 is a schematic illustrating an elongated body with a selectively positioned coating as disclosed and described herein.

FIG. 8 is a sectional view through a longitudinal axis of an elongated body illustrating a selectively positioned coating having defined structural contouring as disclosed and described herein.

FIG. 9 is a schematic illustrating selective positional coating of a plurality of elongated bodies using a microfluidic coating apparatus as disclosed and described herein.

FIG. 10 is a schematic illustrating multiple selective positional coating of a plurality of elongated bodies using a microfluidic coating apparatus as disclosed and described herein.

FIG. 11A is a schematic illustrating an uncoated analyte sensor body as disclosed and described herein.

FIG. 11B is a schematic illustrating an analyte sensor body having a selectively positioned coating with defined structural contouring as disclosed and described herein.

FIG. 12A is a schematic illustrating an uncoated analyte sensor body with a distal secondary electrode as disclosed and described herein.

FIG. 12B is a schematic illustrating the analyte sensor body of FIG. 12A having a selectively positioned coating as disclosed and described herein.

FIG. 12C is a schematic illustrating an analyte sensor body of FIG. 12A having multiple selectively positioned coatings as disclosed and described herein.

FIG. 13A is a schematic illustrating an analyte sensor body with a distal secondary electrode having selectively positioned coating and distal tip coating as disclosed and described herein.

FIG. 13B is a schematic illustrating the analyte sensor body of FIG. 13A having multiple selectively positioned coatings and distal tip coating as disclosed and described herein.

FIG. 14A is a sectional view along a longitudinal axis of the analyte sensor body illustrating a selectively positioned meniscus coating having defined structural contouring as disclosed and described herein.

FIG. 14B is a sectional view along a longitudinal axis of the analyte sensor body illustrating a selectively positioned microfluidic coating having defined structural contouring as disclosed and described herein.

FIG. 15 is a schematic illustrating multiple selective positional coating of a planar analyte sensor body using an array coating apparatus as disclosed and described herein.

FIG. 16 is a front and back side view of a planar multi-analyte sensor body for coating with the apparatuses and methods disclosed herein.

FIG. 17 is a schematic illustrating an elongated analyte sensor body with an array of sensors.

FIG. 18A is a schematic illustrating an elongated analyte sensor body having a photon-activating layer and drug releasing layer.

FIG. 18B is a schematic illustrating a membrane stack including a photon-activating layer and drug releasing layer.

FIG. 18C is a schematic illustrating a membrane stack including a photon-activating layer and drug releasing layer after photon exposure.

FIG. 19 is a diagram illustrating certain embodiments of an example continuous transcutaneous analyte sensor system communicating with at least one display device in accordance with various technologies described in the present disclosure.

### DETAILED DESCRIPTION

The following description and examples illustrate a preferred example of the present disclosure in detail. Those of skill in the art will recognize that there are numerous variations and modifications of this disclosure that are encompassed by its scope. Accordingly, the description of an example should not be deemed to limit the scope of the present disclosure.

In order to facilitate an understanding of the disclosed examples, a number of terms are defined below.

### Definitions

The term “about” as used herein is a broad term, and is to be given its ordinary and customary meaning to a person of ordinary skill in the art (and is not be limited to a special or customized meaning), and refers without limitation to allowing for a degree of variability in a value or range, for example, within 10%, within 5%, or within 1% of a stated value or of a stated limit of a range, and includes the exact stated value or range. The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The phrase “substantially free of” as used herein can mean having none or having a trivial amount of, such that the amount of material present does not affect the material properties of the composition including the material, such that about 0 wt. % to about 5 wt. % of the composition is the material, or about 0 wt. % to about 1 wt. %, or about 5 wt. % or less, or less than or equal to about 4.5 wt. %, 4, 3.5, 3, 2.5, 2, 1.5, 1, 0.9, 0.8, 0.7, 0.6, 0.5, 0.4, 0.3, 0.2, 0.1, 0.1, or about 0.001 wt. % or less, or about 0 wt. %.

The term “adhere” and “attach” as used herein are broad terms, and are to be given their ordinary and customary meaning to a person of ordinary skill in the art (and are not be limited to a special or customized meaning), and refer without limitation to hold, bind, or stick, for example, by gluing, bonding, grasping, interpenetrating, or fusing.

The terms “analyte” as used herein is a broad term, and is to be given its ordinary and customary meaning to a person of ordinary skill in the art (and is not to be limited to a special or customized meaning), and refers without limitation to a substance or chemical constituent in a biological fluid (e.g., blood, interstitial fluid, cerebral spinal fluid, lymph fluid, urine, sweat, saliva, etc.) that can be analyzed. Analytes can include naturally occurring substances, artificial substances, drugs, toxins, metabolites, and/or reaction products. Exemplary analytes include troponin, BNP, insulin, GLP-1, dopamine, serotonin, and L-DOPA.

The phrases “analyte-measuring device,” “analyte-monitoring device,” “analyte-sensing device,” “continuous analyte sensing device,” “continuous analyte sensor device,” and/or “multi-analyte sensor device” as used herein are broad phrases, and are to be given their ordinary and customary meaning to a person of ordinary skill in the art (and are not to be limited to a special or customized meaning), and refer without limitation to an apparatus and/or system responsible for the detection of, or transduction of a signal associated with, a particular analyte, or combination of analytes. For example, these phrases may refer without limitation to an instrument responsible for detection of a particular analyte or combination of analytes. In examples, the instrument includes a sensor coupled to circuitry disposed within a housing, and configure to process signals associated with analyte concentrations into information. In examples, such apparatuses and/or systems are capable of providing specific quantitative, semi-quantitative, qualitative, and/or semi qualitative analytical information using a biological recognition element combined with a transducing and/or detecting element.

The phrase and term “bioactive agent” and “bioactive” as used herein is a broad phrase and a broad term, and are to be given their ordinary and customary meaning to a person of ordinary skill in the art (and is not to be limited to a special or customized meaning), and refers without limitation to any substance that has an effect on or elicits a response from living tissue, for example, drugs, biologics, reactive oxygen scavenger (ROS), and metal ions.

The phrases “biointerface membrane,” “biointerface domain,” and “biointerface layer” as used interchangeably herein are broad phrases, and are to be given their ordinary and customary meaning to a person of ordinary skill in the art (and are not to be limited to a special or customized meaning), and refer without limitation to a permeable membrane (which can include multiple domains) or layer that functions as a bioprotective interface between host tissue and an implantable device. The terms “biointerface” and “bioprotective” are used interchangeably herein.

The terms “biosensor” and/or “sensor” as used herein are broad terms and are to be given their ordinary and customary meaning to a person of ordinary skill in the art (and are not to be limited to a special or customized meaning), and refer without limitation to a part of an analyte measuring device, analyte-monitoring device, analyte sensing device, continuous analyte sensing device, continuous analyte sensor device, and/or multi-analyte sensor device responsible for the detection of, or transduction of a signal associated with, a particular analyte or combination of analytes. In examples, the biosensor or sensor generally comprises a body, a working electrode (WE), a reference electrode (RE), and/or a counter electrode (CE) coupled to body and forming surfaces configured to provide signals during electrochemically reactions. One or more membranes can be affixed to the body and cover electrochemically reactive surfaces. In examples, such biosensors and/or sensors are capable of providing specific quantitative, semi-quantitative, qualitative, semi qualitative analytical signals using a biological recognition element combined with a detecting and/or transducing element.

The term “biostable” as used herein is a broad term, and is to be given its ordinary and customary meaning to a person of ordinary skill in the art (and is not to be limited to a special or customized meaning), and refers without limitation to materials that are relatively resistant to degradation by processes that are encountered in vivo.

The term “co-adsorbate” as used herein is a broad term, and is to be given its ordinary and customary meaning to a person of ordinary skill in the art (and is not to be limited to a special or customized meaning), and refers without limitation to materials that absorb, associate, or couple via covalent, ionic, or molecular interaction to a substrate surface (absorbent).

The term “comprising” as used herein is synonymous with “including,” “containing,” or “characterized by,” and is inclusive or open-ended and does not exclude additional, unrecited elements or method steps.

The term “continuous” as used herein is a broad term, and is to be given its ordinary and customary meaning to a person of ordinary skill in the art (and is not to be limited to a special or customized meaning), and refers without limitation to an uninterrupted or unbroken portion, domain, coating, or layer.

The phrase “continuous analyte sensing” as used herein is a broad phrase, and is to be given its ordinary and customary meaning to a person of ordinary skill in the art (and is not to be limited to a special or customized meaning), and refers without limitation to the period in which monitoring of an analyte concentration is continuously, continually, and/or intermittently (but regularly) performed, for example, from about every 5 seconds or less to about 10 minutes or more. In further examples, continuous monitoring of analyte concentration is performed from about every 10, 15, 20, 25, 30, 35, 40, 45, 50, 55, or 60 second to about 1.25, 1.50, 1.75, 2.00, 2.25, 2.50, 2.75, 3.00, 3.25, 3.50, 3.75, 4.00, 4.25, 4.50, 4.75, 5.00, 5.25, 5.50, 5.75, 6.00, 6.25, 6.50, 6.75, 7.00, 7.25, 7.50, 7.75, 8.00, 8.25, 8.50, 8.75, 9.00, 9.25, 9.50 or 9.75 minutes. In further examples, continuous monitoring of analyte concentration is performed daily and can be performed for weeks.

The term “coupled” as used herein is a broad term, and is to be given its ordinary and customary meaning to a person of ordinary skill in the art (and is not to be limited to a special or customized meaning), and refers without limitation to two or more system elements or components that are configured to be at least one of electrically, mechanically, thermally, operably, chemically or otherwise attached. Similarly, the phrases “operably connected”, “operably linked”, and “operably coupled” as used herein may refer to one or more components linked to another component(s) in a manner that facilitates transmission of at least one signal between the components. In some examples, components are part of the same structure and/or integral with one another (i.e. “directly coupled”). In other examples, components are connected via remote means. For example, one or more electrodes can be used to detect an analyte in a sample and convert that information into a signal; the signal can then be transmitted to an electronic circuit. In this example, the electrode is “operably linked” to the electronic circuit. The phrase “removably coupled” as used herein may refer to two or more system elements or components that are configured to be or have been electrically, mechanically, thermally, operably, chemically, or otherwise attached and detached without damaging any of the coupled elements or components. The phrase “permanently coupled” as used herein may refer to two or more system elements or components that are configured to be or have been electrically, mechanically, thermally, operably, chemically, or otherwise attached but cannot be uncoupled without damaging at least one of the coupled elements or components.

The term “discontinuous” as used herein is a broad term, and is to be given its ordinary and customary meaning to a person of ordinary skill in the art (and is not to be limited to a special or customized meaning), and refers without limitation to disconnected, interrupted, or separated portions, layers, coatings, or domains.

The term “distal” as used herein is a broad term, and is to be given its ordinary and customary meaning to a person of ordinary skill in the art (and is not to be limited to a special or customized meaning), and refers without limitation to a region spaced relatively far from a point of reference, such as an origin or a point of attachment.

The term “domain” as used herein is a broad term, and is to be given its ordinary and customary meaning to a person of ordinary skill in the art (and is not to be limited to a special or customized meaning), and refers without limitation to a region of the membrane system that can be a layer, a uniform or non-uniform gradient (for example, an anisotropic region of a membrane), or a portion of a membrane that is capable of sensing one, two, or more analytes. The domains discussed herein can be formed as a single layer, as two or more layers, as pairs of bi-layers, or as combinations thereof.

The term “drift” as used herein is a broad term, and is to be given its ordinary and customary meaning to a person of ordinary skill in the art (and is not to be limited to a special or customized meaning), and refers without limitation to a progressive increase or decrease in signal over time that is unrelated to changes in host systemic analyte concentrations. While not wishing to be bound by theory, it is believed that drift may be the result of a local decrease in analyte transport to the sensor, for example, due to a formation of a foreign body capsule (FBC). It is also believed that an insufficient amount of interstitial fluid surrounding the sensor may result in reduced transport to the sensor. In examples, an increase in local interstitial fluid may slow or reduce drift and thus improve sensor performance. Drift may also be the result of sensor electronics, or algorithmic models used to compensate for noise or other anomalies that can occur with electrical signals in ranges including the milliampere range, microampere range, picoampere range, nanoampere range, and femtoampere range, likewise with faradic, capacitance, and voltage measurements.

The phrases “bioactive releasing membrane” and “drug releasing layer” and “bioactive releasing domain” and “bioactive agent releasing membrane” are used interchangeably herein and are each a broad phrase, and each are to be given its ordinary and customary meaning to a person of ordinary skill in the art (and is not to be limited to a special or customized meaning), and refers without limitation to a permeable or semi-permeable membrane which is permeable to one or more bioactive agents. In examples, the “bioactive releasing membrane” and “drug releasing layer” and “bioactive releasing domain” and “bioactive agent releasing membrane” can be comprised of two or more domains and is typically of a few microns thickness or more. In examples the bioactive releasing membrane and/or bioactive releasing membrane and/or bioactive agent releasing membrane and/or and bioactive agent releasing membrane are substantially the same as the biointerface layer and/or biointerface membrane. In other examples, the bioactive releasing membrane and/or bioactive releasing membrane and/or bioactive agent releasing membrane and/or and bioactive agent releasing membrane are distinct from the biointerface layer and/or biointerface membrane.

The term “electrochemically reactive surface” as used herein is a broad term, and is to be given its ordinary and customary meaning to a person of ordinary skill in the art (and is not to be limited to a special or customized meaning), and refers without limitation to the surface of an electrode where an electrochemical reaction takes place. In other examples, electron transfer is provided using a redox moiety associated with an aptamer conjugate, where the redox moiety is capable of undergoing reduction-oxidation (redox) that is related to a reversible binding interaction of the aptamer and an analyte proportional to the analyte concentration.

The term “gain” as used herein is a broad term, and is to be given its ordinary and customary meaning to a person of ordinary skill in the art (and is not to be limited to a special or customized meaning), and refers without limitation to a differential measure between signal OFF state and signal ON state. For example, a typical range of gain is 1-200% of a signal percentage change produced by analyte of certain concentration as compared to zero analyte concentration. Analyte concentration is typically quantified in micromolar (uM), nanomolar (nM), nanograms/milliliter (ng/ml) or picograms/milliliter (pg/mL).

The phrase “hard segment” as used herein is a broad phrase, and is to be given its ordinary and customary meaning to a person of ordinary skill in the art (and is not to be limited to a special or customized meaning), and refers without limitation to an element of a copolymer, for example, a polyurethane, a polycarbonate polyurethane, or a polyurethane urea copolymer, which imparts resistance properties, e.g., resistance to bending or twisting. The term “hard segment” can be further characterized as a crystalline, semi-crystalline, or glassy material with a glass transition temperature (Tg) determined by dynamic scanning calorimetry (DSC) typically above ambient temperature, and is typically made of diisocyanate with or without chain extender.

The term “host” as used herein is a broad term, and is to be given its ordinary and customary meaning to a person of ordinary skill in the art (and is not to be limited to a special or customized meaning), and refers without limitation to mammals, for example humans.

The terms “implanted” or “implantable” as used herein are broad terms, and are to be given their ordinary and customary meaning to a person of ordinary skill in the art (and are not to be limited to a special or customized meaning), and refer without limitation to objects (e.g., sensors) that are inserted subcutaneously (i.e. in the layer of fat between the skin and the muscle) or transcutaneously (i.e. penetrating, entering, or passing through intact skin), which may result in a sensor that has an in vivo portion and an ex vivo portion.

The terms “interferants” and “interfering species” as used herein are broad terms, and are to be given their ordinary and customary meaning to a person of ordinary skill in the art (and are not to be limited to a special or customized meaning), and refer without limitation to effects and/or species that interfere with the measurement of an analyte of interest in a sensor to produce a signal that does not accurately represent the analyte measurement. In examples of an electrochemical aptamer sensor, interfering species are compounds with a redox (reduction-oxidation) potential that overlaps with the analyte to be measured or one or more redox moieties associated with one or more aptamers.

The term “in vivo” as used herein is a broad term, and is to be given its ordinary and customary meaning to a person of ordinary skill in the art (and is not to be limited to a special or customized meaning), and without limitation is inclusive of the portion of a device (for example, a sensor) adapted for insertion into and/or existence within a living body of a host.

The term “ex vivo” as used herein is a broad term, and is to be given its ordinary and customary meaning to a person of ordinary skill in the art (and is not to be limited to a special or customized meaning), and without limitation is inclusive of a portion of a device (for example, a sensor) adapted to remain and/or exist outside of a living body of a host.

The term “linker” as used herein is a broad term, and is to be given its ordinary and customary meaning to a person of ordinary skill in the art (and is not to be limited to a special or customized meaning), and without limitation is inclusive of a chemical group or a molecule linking two molecules or moieties. In examples, the linker is positioned between, or flanked by, two groups, molecules, or other moieties and connected to each one via a covalent bond, thus connecting the two. In examples, the linker is an oligonucleotide, biotin, maleimide (NHS) esters, polyethylene glycol-NHS esters, or a “click” chemistry component.

The term “membrane” as used herein is a broad term, and is to be given its ordinary and customary meaning to a person of ordinary skill in the art (and is not to be limited to a special or customized meaning), and refers without limitation to a structure configured to perform functions including, but not limited to, protection of the exposed electrode surface from the biological environment, diffusion resistance (limitation) of the analyte, service as a matrix for a catalyst for enabling an enzymatic reaction, limitation or blocking of interfering species, provision of hydrophilicity at the electrochemically reactive surfaces of the sensor interface, service as an interface between host tissue and the implantable device, modulation of host tissue response via drug (or other substance) release, and combinations thereof. When used herein, the terms “membrane” and “matrix” are meant to be interchangeable.

The phrase “membrane system” as used herein is a broad phrase, and is to be given its ordinary and customary meaning to a person of ordinary skill in the art (and is not to be limited to a special or customized meaning), and refers without limitation to a permeable or semi-permeable membrane that can be comprised of two or more domains, layers, or layers within a domain, and is typically constructed of materials of a few microns thickness or more, which is permeable to analyte. In examples, the membrane system comprises an immobilized or encapsulated aptamer, which enables transduction to occur between the aptamer and analyte whereby a concentration of analyte can be measured.

The term “micro,” as used herein is a broad term, and is to be given its ordinary and customary meaning to a person of ordinary skill in the art (and is not to be limited to a special or customized meaning), and refers without limitation to a small object or scale of approximately 10-6 m that is not visible without magnification. The term “micro” is in contrast to the term “macro,” which refers to a large object that may be visible without magnification. Similarly, the term “nano” refers to a small object or scale of approximately 10-9 m.

The term “noise,” as used herein, is a broad term and is used in its ordinary sense, including, without limitation, a signal detected by the sensor or sensor electronics that is unrelated to analyte concentration and can result in reduced sensor performance. One type of noise has been observed during the few hours (e.g., about 2 to about 24 hours) after sensor insertion. After the first 24 hours, the noise may disappear or diminish, but in some hosts, the noise may last for about three to four days. In some cases, noise can be reduced using predictive modeling, artificial intelligence, and/or algorithmic means. In other cases, noise can be reduced by addressing immune response factors associated with the presence of the implanted sensor, such as using a bioactive releasing membrane with at least one bioactive agent. For example, noise of one or more exemplary biosensors as presently disclosed can be determined and then compared qualitatively or quantitatively. By way of example, by obtaining a raw signal timeseries with a fixed sampling interval (in units of picoampere (pA)), a smoothed version of the raw signal timeseries can be obtained, e.g., by applying a 3rd order lowpass digital Chebyshev Type II filter. Others smoothing algorithms can be used. At each sampling interval, an absolute difference, in units of pA, can be calculated to provide a smoothed timeseries. This smoothed timeseries can be converted into units (the unit of “noise”), using, for example, an analyte sensitivity timeseries, where the analyte sensitivity timeseries is derived by using a mathematical model between the raw signal and reference blood analyte measurements. Optionally, the timeseries can be aggregated as desired, e.g., by hour or day. Comparison of corresponding timeseries between different exemplary biosensors with the presently disclosed bioactive releasing membrane and one or more bioactive agents provides for qualitative or quantitative determination of improvement of noise.

The term “optional” or “optionally” as used herein is a broad term, and is to be given its ordinary and customary meaning to a person of ordinary skill in the art (and is not to be limited to a special or customized meaning), and, without limitation, means that the subsequently described event or circumstance can or cannot occur, and that the description includes instances where the event or circumstance occurs and instances where it does not.

The phrase “polymerization group” used herein is a broad phrase, and is to be given its ordinary and customary meaning to a person of ordinary skill in the art (and is not to be limited to a special or customized meaning), and refers without limitation to a functional group that permits polymerization of the monomer with itself to form a homopolymer or together with different monomers to form a copolymer. Depending on the type of polymerization methods employed, the polymerization group can be selected from alkene, alkyne, epoxide, lactone, amine, hydroxyl, isocyanate, carboxylic acid, anhydride, silane, halide, aldehyde, and carbodiimide.

The term “polyzwitterions” as used herein is a broad term, and is to be given its ordinary and customary meaning to a person of ordinary skill in the art (and is not to be limited to a special or customized meaning), and refers without limitation to polymers where a repeating unit of the polymer chain is a zwitterionic moiety. Polyzwitterions are also known as polybetaines. Since polyzwitterions have both cationic and anionic groups, they are a type of polyampholytic polymer. They are unique, however, because the cationic and anionic groups are both part of the same repeating unit, which means a polyzwitterion has the same number of cationic groups and anionic groups whereas other polyampholytic polymers can have more of one ionic group than the other. Also, polyzwitterions have the cationic group and anionic group as part of a repeating unit. Polyampholytic polymers need not have cationic groups connected to anionic groups; they can be on different repeating units and thus may be distributed apart from one another at random intervals, or one ionic group may outnumber the other.

The term “proximal” as used herein is a broad term, and is to be given its ordinary and customary meaning to a person of ordinary skill in the art (and is not to be limited to a special or customized meaning), and refers without limitation to the spatial relationship between various elements in comparison to a particular point of reference. For example, some examples of a device include a membrane system having a biointerface layer and an enzyme layer. If the sensor is deemed to be the point of reference and the enzyme layer is positioned nearer to the sensor than the biointerface layer, then the enzyme layer is more proximal to the sensor than the biointerface layer.

The phrase and term “processor module” and “microprocessor” as used herein are each a broad phrase and term, and are to be given their ordinary and customary meaning to a person of ordinary skill in the art (and are not to be limited to a special or customized meaning), and refer without limitation to a computer system, state machine, processor, or the like designed to perform arithmetic or logic operations using logic circuitry that responds to and processes the basic instructions that drive a computer.

The term “semi-continuous” as used herein is a broad term, and is to be given its ordinary and customary meaning to a person of ordinary skill in the art (and is not to be limited to a special or customized meaning), and refers without limitation to a portion, coating, domain, or layer that includes one or more continuous and noncontinuous portions, coatings, domains, or layers. For example, a coating disposed around a sensing region but not about the sensing region is “semi-continuous.”

The phrases “sensing portion,” “sensing membrane,” “sensing region,” “sensing domain,” and/or “sensing mechanism” as used herein are broad phrases, and are to be given their ordinary and customary meaning to a person of ordinary skill in the art (and are not to be limited to a special or customized meaning), and refer without limitation to the part of a biosensor and/or a sensor responsible for the detection of, or transduction of a signal associated with, a particular analyte or combination of analytes. In examples, the sensing portion, sensing membrane, and/or sensing mechanism generally comprise an electrode configured to provide signals during electrochemically reactions with one or more membranes covering electrochemically reactive surface. In examples, such sensing portions, sensing membranes, and/or sensing mechanisms are capable of providing specific quantitative, semi-quantitative, qualitative, semi qualitative analytical signals using a biological recognition element combined with a detecting and/or transducing element.

In examples, the sensing region or sensing portion can comprise at least a portion of a conductive substrate or at least a portion of a conductive surface, for example, a wire or conductive trace or a substantially planar substrate including substantially planar trace(s), and a membrane. In examples, the sensing region or sensing portion can comprise a non-conductive body, a working electrode, a reference electrode, and a counter electrode (optional), forming an electrochemically reactive surface at one location on the body and an electronic connection at another location on the body, and a sensing membrane affixed to the body and covering the electrochemically reactive surface.

In examples, multiple working electrodes can be employed. For example, a second working electrode comprising a plurality of different analyte (e.g., analyte 1, analyte2, etc.) aptamer conjugates on the second working electrode to correct for sensor drift and/or interference. Likewise, a second working electrode comprising a non-selective aptamer conjugate to a plurality of different analytes (e.g., analyte 1, analyte2, etc.) on the second working electrode can be used to correct for sensor drift and/or interference.

In other examples, the sensing region can comprise one or more periplasmic binding protein (PBP) or mutant or fusion protein thereof having one or more analyte binding regions, each region capable of specifically and reversibly binding to at least one analyte. Mutations of the PBP can contribute to or alter one or more of the binding constants, extended stability of the protein, including thermal stability, to bind the protein to a special encapsulation matrix, membrane or polymer, or to attach a detectable reporter group or “label” to indicate a change in the binding region. Specific examples of changes in the binding region include, but are not limited to, hydrophobic/hydrophilic environmental changes, three-dimensional conformational changes, changes in the orientation of amino acid side chains in the binding region of proteins, and redox states of the binding region. Such changes to the binding region provide for transduction of a detectable signal corresponding to the one or more analytes present in the biological fluid.

In examples, the sensing region determines the selectivity among one or more analytes, so that only the analyte which has to be measured leads to (transduces) a detectable signal. The selection may be based on any chemical or physical recognition of the analyte by the sensing region, where the chemical composition of the analyte is unchanged, or in which the sensing region causes or catalyzes a reaction of the analyte that changes the chemical composition of the analyte.

The sensing region transduces the recognition of analytes into a semi-quantitative or quantitative signal. Thus, “transducing” or “transduction” and their grammatical equivalents as are used herein encompasses optical, electrochemical, acoustical/mechanical, or colorimetrical technologies and methods. Electrochemical properties include current and/or voltage, capacitance, and potential. Optical properties include absorbance, fluorescence/phosphorescence, wavelength shift, phase modulation, bio/chemiluminescence, reflectance, light scattering, and refractive index.

The term “sensitivity” as used herein is a broad term, and is to be given its ordinary and customary meaning to a person of ordinary skill in the art (and is not to be limited to a special or customized meaning), and refers without limitation to an amount of signal (e.g., in the form of electrical current and/or voltage) produced by a predetermined amount (unit) of the measured analyte. For example, an amperometric sensor has a sensitivity (or slope) of from about 1 to about 100 picoAmps of current for every 1 mg/dL of analyte.

The phrases and terms “small diameter sensor,” “small structured sensor,” and “micro-sensor” as used herein are broad phrases and terms, and are to be given their ordinary and customary meaning to a person of ordinary skill in the art (and are not to be limited to a special or customized meaning), and refer without limitation to sensing mechanisms that are less than about 2 mm in at least one dimension. In further examples, the sensing mechanisms are less than about 1 mm in at least one dimension. In some examples, the sensing mechanism (sensor) is less than about 0.95, 0.9, 0.85, 0.8, 0.75, 0.7, 0.65, 0.6, 0.5, 0.4, 0.3, 0.2, or 0.1 mm. In some examples, the maximum dimension of an independently measured length, width, diameter, thickness, or circumference of the sensing mechanism does not exceed about 2 mm. In some examples, the sensing mechanism is a needle-type sensor, wherein the diameter is less than about 1 mm, see, for example, U.S. Pat. No. 6,613,379 to Ward et al. and U.S. Pat. No. 7,497,827 to Brister et al., both of which are incorporated herein by reference in their entirety. In some alternate examples, the sensing mechanism includes electrodes deposited on a substantially planar substrate, wherein the thickness of the implantable portion is less than about 1 mm, see, for example U.S. Pat. No. 6,175,752 to Say et al. and U.S. Pat. No. 5,779,665 to Mastrototaro et. al., both of which are incorporated herein by reference in their entirety. Examples of methods of forming the sensors (sensor electrode layouts and membrane) and sensor systems discussed herein may be found in currently pending U.S. Pat. Pub. No. 2019-0307371, which is incorporated by reference in its entirety herein.

The phrase “soft segment” as used herein is a broad phrase, and is to be given its ordinary and customary meaning to a person of ordinary skill in the art (and is not to be limited to a special or customized meaning), and refers without limitation to an element of a copolymer, for example, a polyurethane, a polycarbonate polyurethane, or a polyurethane urea copolymer, which imparts flexibility to the chain. The phrase “soft segment” can be further characterized as an amorphous material with a low Tg, e.g., a Tg not typically higher than ambient temperature or normal mammalian body temperature.

The phrase “solid portions” as used herein is a broad term, and is to be given its ordinary and customary meaning to a person of ordinary skill in the art (and is not to be limited to a special or customized meaning), and refers without limitation to portions of a membrane's material having a mechanical structure that demarcates cavities, voids, or other non-solid portions.

The term and phrases “zwitterion” and “zwitterionic compound” as used herein are each a broad term and phrase, and are to be given their ordinary and customary meaning to a person of ordinary skill in the art (and is not to be limited to a special or customized meaning), and refer without limitation to compounds in which a neutral molecule of the compound has a unit positive and unit negative electrical charge at different locations within the molecule. Such compounds are a type of dipolar compound, and are also sometimes referred to as “inner salts.”

The phrases “zwitterion precursor” or “zwitterionic compound precursor” as used herein are broad phrases, and are to be given their ordinary and customary meaning to a person of ordinary skill in the art (and is not to be limited to a special or customized meaning), and refer without limitation to any compound that is not itself a zwitterion, but can become a zwitterion in a final or transition state through chemical reaction. In some examples described herein, devices comprise zwitterion precursors that can be converted to zwitterions prior to in vivo implantation of the device. Alternately, in some examples described herein, devices comprise zwitterion precursors that can be converted to zwitterions by some chemical reaction that occurs after in vivo implantation of the device. Such reactions are known to a person of ordinary skill in the art and include ring opening reaction, addition reaction such as Michael addition. This method is especially useful when the polymerization of betaine containing monomer is difficult due to technical challenges such as solubility of betaine monomer to achieve desired physical properties such as molecular weight and mechanical strength. Post-polymerization modification or conversion of betaine precursor can be a practical way to achieve desired polymer structure and composition. Examples of such as precursors include tertiary amines, quaternary amines, pyridines, and others detailed herein.

The phrases “zwitterion derivative” or “zwitterionic compound derivative” as used herein are broad phrases, and are to be given their ordinary and customary meaning to a person of ordinary skill in the art (and is not to be limited to a special or customized meaning), and refer without limitation to any compound that is not itself a zwitterion, but rather is the product of a chemical reaction where a zwitterion is converted to a non-zwitterion. Such reactions can be reversible, such that under certain conditions zwitterion derivatives can act as zwitterion precursors. For example, hydrolysable betaine esters formed from zwitterionic betaines are cationic zwitterion derivatives that under the appropriate conditions are capable of undergoing hydrolysis to revert to zwitterionic betaines.

The phrases “zwitterionic repeating group” as used herein is a broad phrase, and is to be given their ordinary and customary meaning to a person of ordinary skill in the art (and is not to be limited to a special or customized meaning), and refer without limitation to, independently, two or more zwitterionic compounds, zwitterion derivatives or zwitterionic compound derivatives in the same compound or polymer.

Coatings applied to a sensor or an elongated body are generally described herein after the coating has dried or cured on the sensor or an elongated body, unless noted otherwise. Coatings can improve the sensor function and mitigate degradation during in vivo use.

One reason for functional degradation of Continuous Glucose Monitors (CGM's) is the immunological response to the implanted portion thereof, which results in a progressive degeneration of the signal during operation. For implantable sensors with electrochemically active sites, a hydrophobic coating layer containing a releasable active agent, for example, proximal to an in vivo electrochemically active site of the implantable sensor has been shown to suppress/inhibit the immunological response and extend the end-of-life (EOL) of the implantable sensor, however, close proximity of the hydrophobic coating layer containing a releasable active agent to the active site may result in measurable drift and lag time response.

Devices and probes that are transcutaneously inserted or implanted into subcutaneous tissue can elicit a foreign body response (FBR), which includes invasion of inflammatory cells that ultimately forms a foreign body capsule (FBC), as part of the body's response to the introduction of a foreign material. Continuous monitoring systems discussed herein include continuous analyte monitoring systems configured to monitor one, two, or more analytes concurrently, sequentially, and/or randomly (which is inclusive of events that can take place independently in picoseconds, nanoseconds, milliseconds, seconds, or minutes) to predict health-related events and health systems performance (e.g., the current and future performance of the human body's systems such as the circulatory, respiratory, digestive, or other systems or combinations of organs or systems). In examples, insertion or implantation of a device, for example, a glucose sensing device, can result in an acute inflammatory reaction resolving to chronic inflammation with concurrent building of fibrotic tissue, such as described in detail above. Eventually, over a period of time, a mature FBC, including primarily contractile fibrous tissue forms around the device. See Shanker and Greisler, Inflammation and Biomaterials in Greco RS, ed., “Implantation Biology: The Host Response and Biomedical Devices” pp 68-80, CRC Press (1994). The FBC surrounding conventional implanted devices has been shown to hinder or block the transport of analytes across the device-tissue interface. Thus, continuous extended life analyte transport (e.g., beyond the first few days) in vivo has been conventionally believed to be unreliable or impossible.

In some examples, certain aspects of the FBR in the first few days may play a role in noise. It has been observed that some sensors comprising drug releasing layers function differently during the first few hours after insertion than they do later in a wear period. This is exemplified by noise and/or a suppression of the signal during the first few hours (e.g., about 2 to about 24 hours) after insertion. These anomalies often resolve spontaneously after which the sensors become less noisy, more responsive, have improved sensitivity, and are more accurate than during the early wear period.

Without being bound by any theory, it is believed that this change in performance of device function is most likely due to certain drugs released from the drug releasing layer that migrate to tissue near the sensor site during the first few minutes/hours after implantation and reduce analyte transport to the sensor's working electrode. In some examples, the drug releasing layer coated selectively on a portion of the sensor provides unique advantages in vivo (e.g., one to 14 days) that can be used delay a drug's effect on the local tissue surrounding the working electrode. Such materials can also provide advantages in the long term too (e.g., greater than 14 days) to enhance and extend sensor performance and lifetime. Particularly, the in vivo portion of the sensor (the portion of the sensor that is implanted into the host's tissue) selectively coated on a portion thereof reduces sensor performance delays upon implantation, such as an initial lag in signal, while extending sensor lifetime to more than 14 days, more than 21 days, and to 28 days.

In some examples, the drug releasing layer comprises a bioactive agent that releases upon insertion. In examples, the drug releasing layer allows drug release/elution over an extended period of time. In examples, as the bioactive agent releases/elutes from the drug releasing layer, transport of the analyte through the tissue may be delayed from the release of the aforementioned bioactive agent and its migration to other membranes and/or portions of the implantable portion of the sensor.

Further examples of drug releasing layers comprising a bioactive agent and membranes may be found in pending U.S. Patent Publication No. 2023/0073214, titled “BIOACTIVE RELEASING MEMBRANE FOR ANALYTE SENSOR,” filed Sep. 15, 2022, incorporated by reference in its entirety herein.

As a result of the coating methods described herein, in examples, foreign body response is reduced or delayed for at least 7 days, at least 14 days, at least 21 days, at least 28 days without a lag in response upon insertion, for example, within the first half hour, hour, or hours after insertion by the selective positioning of a drug releasing coating on the implanted portion of the continuous analyte monitoring sensor.

Continuous analyte sensors typically include multiple layers of materials providing one or more functions to provide a measure of an analyte concentration. In the manufacture of some continuous analyte sensors, various methods are deployed to deposit the multiple layers of materials, for example, by dip-coating. However, in some situations, a dip-coated drug releasing layer (DL or DR as used herein) proximal a distal tip of an implanted portion of an analyte sensor effects initial signal performance. While not to held to any particular theory, hydrophobic drug releasing layers covering the distal tip can attenuation analyte transport to the working electrode.

While it is possible to selectively coat a coating solution on a proximal side of a sensor with dip coating methods, via coating from the proximal side and avoiding exposing skived sections to the solvents, followed by stripping off of unwanted coating using suitable solvent, this method is time consuming and complex, as it involves thin and small elongated objects and involves risk of stripping other membrane(s) during this process as well as resulting in higher variability of the coating metrics i.e., thickness, coat length etc.

As a result of the coating apparatuses and methods described herein, in examples, a drug releasing layer is selectively coated on a portion of the implanted sensor and the foreign body response is reduced or delayed for at least 7 days, at least 14 days, at least 21 days without a lag in response upon insertion, for example, without lag in response within the first half hour, hour, or hours after insertion.

Thus, in examples, selective coating apparatuses and processes are disclosed for positioning a drug releasing layer (DL) distal from the implanted distal tip portion and the working electrode. The selective coating processes disclosed herein can be performed subsequent one or more dip coating processes. The selective coating apparatuses and processes are also envisaged for positioning other membranes or layers of a continuous analyte monitoring device.

For example, one such selective coating process involves one or more meniscus coating methods. In examples, one or both of the meniscus coating methods comprise one or more feedback loops, e.g., to control viscosity, temperature, or flow rate. The one or more meniscus coating methods are configured for wire-based sensors or planar sensors.

In other examples, a selective coating method is used to position a coating solution distal from the implanted distal tip portion, the method comprising one or more microfluidic coating methods. The one or more microfluidic coating methods are configured for wire-based sensors or planar sensors.

The disclosed selective alternate coating apparatuses and methods is directed at applying a coating layer at any desired location on the elongated body of a device, e.g., an analyte sensor. Other elongated body medical devices can be selectively coated using the apparatuses and methods disclosed herein, such as stents, birth control implants, pacemaker leads, catheters, and the like.

### Elongated Body Configurations for Selective Positional Coating

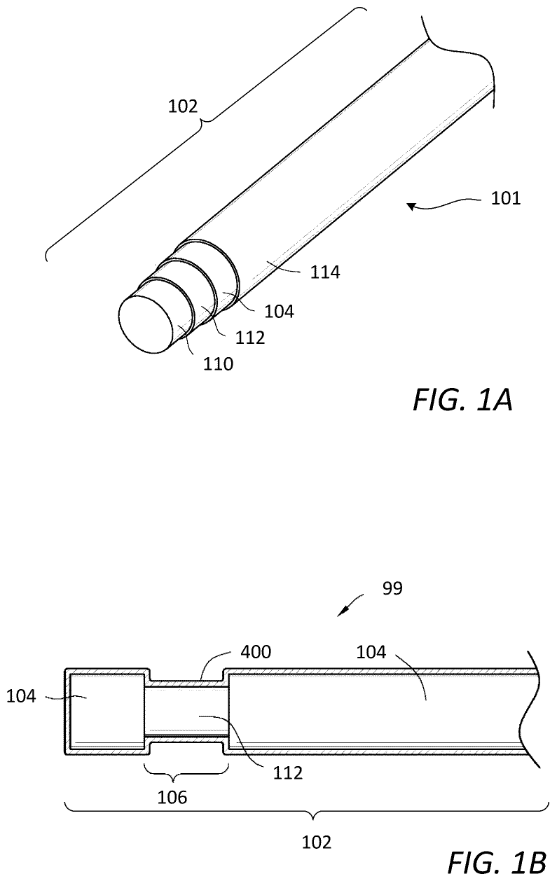

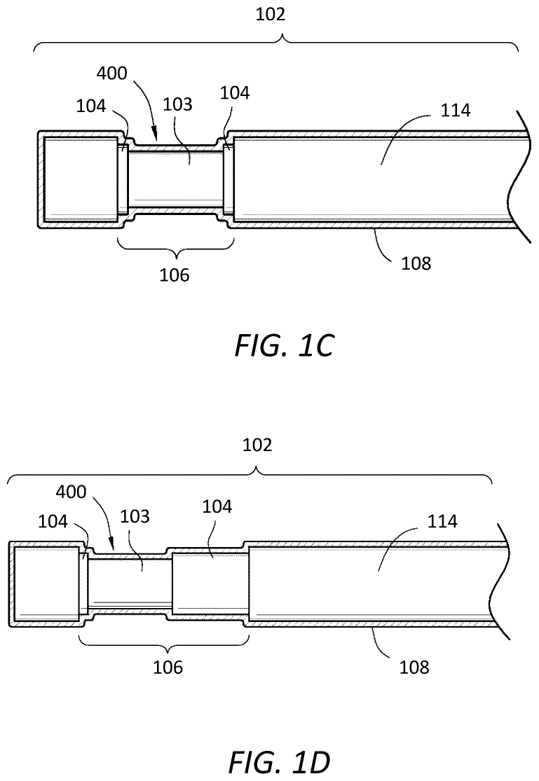

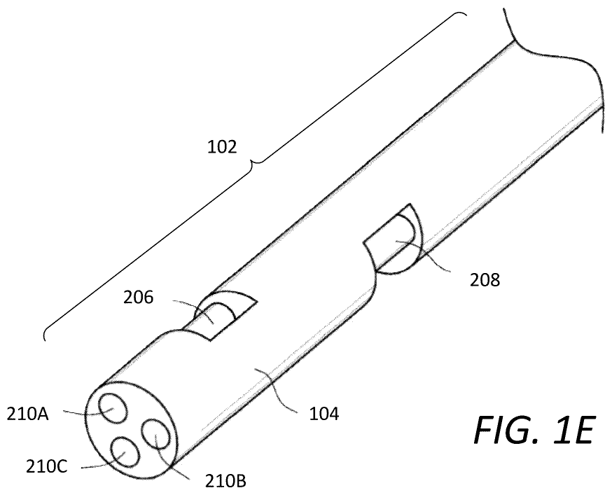

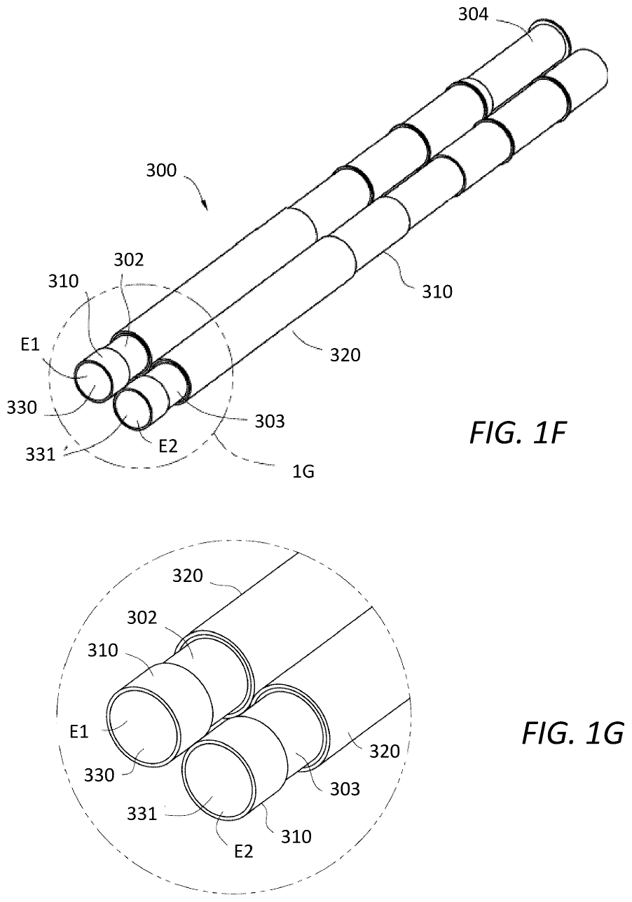

FIGS. 1A through 1D illustrate an exemplary configurations of an in vivo portion of a continuous analyte sensor 100, which includes an elongated conductive body 102. The elongated conductive body 102 includes a core 110 (see FIG. 1B) and a first layer 112 at least partially surrounding the core. The first layer includes a working electrode (for example, located in window 106) and a sensing membrane 400 located over the working electrode. In some examples, the core and first layer can be of a single material (such as, for example, platinum). In some examples, the elongated conductive body is a composite of at least two materials, such as a composite of two conductive materials, or a composite of at least one conductive material and at least one non-conductive material. In some examples, the elongated conductive body comprises a plurality of layers. In certain examples, there are at least two concentric or annular layers, such as a core formed of a first material and a first layer formed of a second material. However, additional layers can be included in some examples. In some examples, the layers are coaxial.

The elongated conductive body can be long and thin, yet flexible and strong. For example, in some examples, the smallest dimension of the elongated conductive body is less than about 0.1 inches (2.54 mm), 0.075 inches (1.90 mm), 0.5 inches (1.27 mm), 0.025 inches (0.635 mm), 0.1 inches (0.254 mm), 0.004 inches (0.1 mm), or 0.002 inches (0.5 mm). While the elongated conductive body is illustrated in FIGS. 1A through 1C as having a circular cross-section, in other examples the cross-section of the elongated conductive body can be planar, ovoid, rectangular, triangular, polyhedral, star-shaped, C-shaped, T-shaped, X-shaped, Y-Shaped, irregular, or the like. In examples, a conductive wire electrode is employed as a core. To such a clad electrode, two additional conducting layers can be added (e.g., with intervening insulating layers provided for electrical isolation). The conductive layers can be comprised of any suitable material. In certain examples, it can be desirable to employ a conductive layer comprising conductive particles (i.e., particles of a conductive material) in a polymer or other binder.

The materials used to form the elongated conductive body (such as, for example, stainless steel, titanium, tantalum, platinum, platinum-iridium, iridium, nitinol, certain polymers, and/or combinations thereof) can be strong and hard, and therefore are resistant to breakage. In some examples, the sensor's small diameter provides flexibility to these materials, and therefore to the sensor as a whole. Thus, the sensor can withstand repeated forces applied to it by surrounding tissue.

In addition to providing structural support, resiliency and flexibility, in some examples, the core 110, or a component thereof, provides electrical conduction for an electrical signal from the working electrode to sensor electronics (not shown). In some examples, the core 110 comprises a conductive material, such as nitinol, stainless steel, titanium, tantalum, a conductive polymer, and/or the like. However, in other examples, the core is formed from a non-conductive material, such as a non-conductive polymer. In yet other examples, the core comprises a plurality of layers of materials. For example, in examples the core includes an inner core and an outer core. In a further example, the inner core is formed of a first conductive material and the outer core is formed of a second conductive material. For example, in some examples, the first conductive material is nitinol, stainless steel, titanium, tantalum, a conductive polymer, an alloy, and/or the like, and the second conductive material is a conductive material selected to provide electrical conduction between the core and the first layer, and/or to attach the first layer to the core (that is, if the first layer is formed of a material that does not attach well to the core material). In other examples, the core is formed of a non-conductive material (such as, for example, a non-conductive metal and/or a non-conductive polymer) and the first layer is formed of a conductive material, such as nitinol, stainless steel, titanium, tantalum, a conductive polymer, and/or the like. The core and the first layer can be of a single (or same) material, such as platinum. One skilled in the art appreciates that additional configurations are possible.

First layer 112 can be formed of a conductive material and the working electrode can be an exposed portion of the surface of the first layer 112. Accordingly, the first layer 112 can be formed of a material configured to provide a suitable electroactive surface for the working electrode, a material such as, but not limited to, platinum, platinum-iridium, gold, palladium, iridium, graphite, carbon, a conductive polymer, an alloy and/or the like.

As illustrated in FIG. 1B, FIG. 1C, and FIG. 1D, second layer 104 surrounds at least a portion of the first layer 112, thereby defining the boundaries of the working electrode. In some examples, the second layer 104 serves as an insulator and is formed of an insulating material, such as polyimide, polyurethane, parylene, or any other known insulating materials. For example, in examples the second layer is disposed on the first layer and configured such that the working electrode is exposed via window 106. In some examples, an elongated conductive body, including the core, the first layer and the second layer, is provided. A portion of the second layer can be removed to form a window 106, through which the electroactive surface of the working electrode (that is, the exposed surface of the first layer 112) is exposed. In some examples, a portion of the second and (optionally) third layers can be removed to form the window 106, thus exposing the working electrode. Removal of coating materials from one or more layers of the elongated conductive body (for example, to expose the electroactive surface of the working electrode) can be performed by hand, excimer lasing, chemical etching, laser ablation, grit-blasting, or the like.

The sensor can further comprise a third layer 114 comprising a conductive material. For example, the third layer 114 can comprise a reference electrode, which can be formed of a silver-containing material that is applied onto the second layer 104 (that is, the insulator).

The elongated conductive body 102 can further comprise one or more intermediate layers (not shown) located between the core 110 and the first layer 112. For example, the intermediate layer can be one or more of an insulator, a conductor, a polymer, and/or an adhesive. In some examples, the core 110 comprises a non-conductive polymer and the first layer 112 comprises a conductive material. Such a sensor configuration can advantageously provide reduced material costs, in that it replaces a typically expensive material with an inexpensive material. For example, the core 110 can be formed of a non-conductive polymer, such as, a nylon or polyester filament, string or cord, which can be coated and/or plated with a conductive material, such as platinum, platinum-iridium, gold, palladium, iridium, graphite, carbon, a conductive polymer, and allows or combinations thereof.

Referring to FIG. 1B and FIG. 1C, the reference electrode 114 can comprise a silver-containing material (e.g., silver/silver chloride) applied over at least a portion of the insulating material 104, as discussed in greater detail elsewhere herein. For example, the silver-containing material can be applied using the presently disclosed coating methods described herein. In examples, the silver-containing material can be applied using the presently disclosed coating methods in combination with thin film and/or thick film techniques, such as but not limited to the methods disclosed herein, dipping, spraying, printing, electro-depositing, vapor deposition, spin coating, and sputter deposition. For example, a silver or silver chloride-containing paint (or similar formulation) can be applied to a reel of the insulated conductive core using the presently disclosed coating methods.

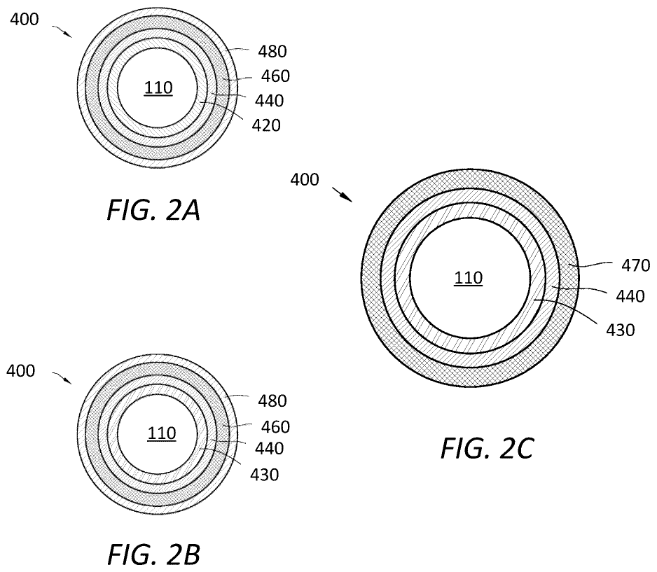

As illustrated in FIG. 1C and FIG. 1D, the sensor can also include a sensing membrane 400, such as those discussed elsewhere herein, for example, with reference to FIGS. 2A through 2C. The sensing membrane 400 can include an enzyme layer (not shown), as described elsewhere herein. For example, the sensing membrane 400 can include a catalyst or enzyme configured to react with an analyte. For example, the sensing membrane 400 can be an immobilized enzyme-containing layer including glucose oxidase, glucose dehydrogenase, β-hydroxybutyrate dehydrogenase, or lactate dehydrogenase. In other examples, the sensing membrane 400 can be impregnated with other oxidases, including, for example, galactose oxidase, cholesterol oxidase, amino acid oxidase, alcohol oxidase, lactate oxidase, aspartate oxidase, or uricase. In examples, the sensing membrane 400 can include one or more of glucose dehydrogenase, lactate dehydrogenase, malate dehydrogenase, glycerol dehydrogenase, alcohol dehydrogenase, sorbitol dehydrogenase, and an amino acid dehydrogenase comprising L-amino acid dehydrogenase, asparaginase, or superoxide dismutase.

In examples, the enzyme layer can include β-hydroxybutyrate dehydrogenase (HBD) enzyme, a nicotinamide adenine dinucleotide (NAD+) and a metal or non-metal mediator. In examples, the sensing membrane 400 comprises β-hydroxybutyrate dehydrogenase (HBD) enzyme, a nicotinamide adenine dinucleotide (NAD+) and a metal or non-metal mediator in combination with glucose oxidase or glucose dehydrogenase.

In examples, the sensing membrane 400 comprises aspartate oxidase and/or asparaginase. In examples, the sensing membrane 400 comprises aspartate oxidase and/or asparaginase in combination with glucose oxidase or glucose dehydrogenase or β-hydroxybutyrate dehydrogenase (HBD) enzyme, a nicotinamide adenine dinucleotide (NAD+) and a metal or non-metal mediator.

Combinations of the above enzymes can be combined in the same layer or provided in separated layers vertically and/or horizontally separated about the elongated body are envisaged with the coating methods disclosed herein. Combinations of the above enzymes can be combined in the same layer or provided in separated layers vertically and/or horizontally separated with one or more intervening layers about the elongated body are envisaged with the coating methods disclosed herein.

FIG. 1E is a perspective view of the in vivo portion of a multi-analyte capable sensor. In this example, the insulated elongated body comprises three conductive cores 210A, 210B, 210C located in (e.g., embedded in, coated with) the insulator 104. In this example, a plurality of windows is formed in and/or through the insulator, such that each window exposes a portion of a different core. In examples, one core is a counter electrode and the other two cores are working electrodes of the same or different conducting material and/or comprising the same or different sensing membrane, e.g., an analyte sensor or a multi-analyte sensor, such as a glucose/ketone sensor, for example. In examples, one core is a counter electrode, one core is a reference electrode, and the remaining core is a working electrode. As a non-limiting example, window 206 is formed in the insulator such that a portion of core 210A is exposed. Similarly, window 208 is formed in the insulator such that a portion of core 210B is exposed. Core 210C, in some examples, is a counter electrode configured to be exposed at the distal tip of the elongated body as shown. The windows can be longitudinally staggered and/or non-staggered along the longitudinal length of the sensor. In examples, one core is a counter electrode configured to be exposed at the distal end of the elongated body, and the other two cores are working electrodes of the same or different conducting material, one with a sensing membrane as described herein, devoid of a drug releasing layer in one window, e.g., window 208, and the other working electrode is devoid of sensing membrane but having a drug releasing layer in window 206.

In examples, the first conductive core is formed of platinum, platinum-iridium, gold, palladium, iridium, graphite, carbon, a conductive polymer and/or an alloy, and a first window 206 is configured and arranged to expose an electroactive portion of the first conductive core. The second conductive core is formed of a silver-containing material (e.g., a silver or silver/silver-chloride wire, or a silver-containing wire-shaped a silver-containing material body), and a second window 208 is configured and arranged to expose an electroactive portion of the second conductive core. In some examples, instead of a bulk metal wire, the first conductive core comprises an inner core and an outer core. For example, to reduce material costs, the inner core is formed of a material that is relatively less expensive than platinum, such as stainless steel, titanium, tantalum and/or a polymer, and the outer core is formed of a material that provides an appropriate electroactive surface, such as but not limited to platinum, platinum-iridium, gold, palladium, iridium, graphite, carbon, a conductive polymer and/or an alloy. In some examples, the membrane covers the exposed electroactive portion of the first conductive core. In a further example, the membrane covers the in vivo portion of the sensor. In some examples, a third conductive core is embedded in the insulator. In some examples, the third conductive core is configured and arranged as a second working electrode, which can be configured as a redundant working electrode, a non-analyte signal-measuring working electrode (e.g., no transducing element as described below), as a counter working electrode, to detect a second analyte, and/or the like.

FIG. 1F is a perspective view of the in vivo portion of an example of a multi-electrode sensor system 300 comprising two working electrodes and at least one reference/counter electrode. The sensor system 300 comprises first and second elongated bodies E1, E2, each formed of a conductive core or of a core with a conductive layer deposited thereon. In this particular example, a wire-based sensor is shown, however, a planar arrangement is also envisaged. In this particular example, an insulating layer 310, a conductive layer 320 e.g., a reference electrode, and any one of the previously described membranes (not shown) are deposited on top of the elongated bodies E1, E2. The insulating layer 310 separates the conductive layer 320 from the elongated body. The materials selected to form the insulating layer 310 may include any of the insulating materials described elsewhere herein, including polyurethane and polyimide. The materials selected to form the conductive layer 320 may include any of the conductive materials described elsewhere herein, including silver/silver chloride, platinum, gold, etc. Working electrodes 302, 303 are formed by removing portions of the conductive layer 320 and the insulating layer 310, thereby exposing electroactive surface of the elongated bodies E1, E2, respectively. FIG. 1G provides a close perspective view of the distal portion of the elongated bodies E1, E2.

In examples, the two elongated bodies illustrated in FIG. 1F are fabricated to have substantially the same shape and dimensions. In examples, the two elongated bodies illustrated in FIG. 1F are fabricated to have substantially the same shape and dimensions but with one elongated body having no enzyme or a deactivated enzyme and a drug releasing layer, whereas the other elongated body includes an active enzyme without a drug releasing layer.

In other examples, the two elongated bodies illustrated in FIG. 1F, but with one elongated body having no window 302 and a drug releasing layer, whereas the other elongated body includes a widow 303 and sensing membrane with an active enzyme without a drug releasing layer.

In some examples, the working electrodes of FIG. 1F are fabricated to have the same properties, thereby providing a sensor system capable of providing redundancy of signal measurements or providing unique signals representing two or more different analytes. In other examples, the working electrodes, associated with the elongated bodies E1, E2, may each have one or more characteristics that distinguish each working electrode from the other. For example, in examples, each of the elongated bodies E1, E2 may be different conductive surfaces, so that each working electrode has a different electrochemical property than the other working electrode. In addition, in examples, each of the elongated bodies E1, E2 may be covered with different membrane(s), so that each working electrode has a different membrane property than the other working electrode.

Although not shown in FIGS. 1F-1G, in certain examples, the exposed distal ends 330, 331 of the core portions of the elongated bodies E1, E2 may be covered with an insulating material (e.g., polyurethane or polyimide). In alternative examples, the exposed distal ends 330, 331 of the core portions are covered with any of the previously described membrane system and/or serve as additional or “secondary” working electrode surface area.

Regarding fabrication of the sensor system illustrated in FIG. 1F-1G, in examples, the elongated bodies E1, E2 may be formed as an elongated conductive core, or alternatively as a core (conductive or non-conductive) having at least one conductive material deposited thereon. Next, an insulating layer 310 is deposited onto each of the elongated bodies E1, E2. Thereafter, a conductive layer 320 is deposited over the insulating layer 310. The conductive layer 320 may serve as a reference/counter electrode and may be formed of silver/silver chloride, or any other material that may be used for a reference electrode. In alternative examples, the conductive layer 320 may be formed of a different conductive material, and may be used another working electrode. After these steps, a layer removal process is performed to remove portions of the deposited layers (i.e., the conductive layer 320 and/or the insulating layer 310). Any of the techniques described elsewhere herein (e.g., laser ablation, chemical etching, grit blasting) may be used. In the example illustrated in FIGS. 1F and 1G, layers of the conductive layer 320 and the insulating layer 310 are removed to form the working electrodes 302, 303. Although in the example shown, layer removal is performed across the entire cross-sectional perimeter (e.g., circumference) of the deposited layer, it is contemplated that in other examples, layer removal may be performed across a preselected section of the cross-sectional perimeter, instead of across the entire cross-sectional perimeter.

Contacts 304 can be used to provide electrical connection between the working electrodes and other components of the sensor system may be formed in a similar manner. As shown, contacts 304 are separated from each other to prevent an electrical connection therebetween. Because the layer removal process is performed on each individual elongated body E1, E2, instead of a single geometrically complicated elongated body, this particular sensor design (i.e., two elongated bodies placed side by side) may provide ease of manufacturing, as compared to the manufacturing processes involved with other multi-electrode systems having other geometries.

After the conductive and insulating layers are deposited onto the elongated body, and after selected portions of the deposited layers have been removed, one or more membranes are applied onto at least a portion of the elongated bodies using the apparatuses and method disclosed herein, either alone or in combination with the apparatuses and method disclosed herein or with other coating apparatuses and methods. In certain examples, any of the aforementioned membrane systems are applied only to the working electrodes, but in other examples any of the aforementioned membrane systems are applied to the entire elongated body. In examples, any of the aforementioned membrane systems are deposited onto the two working electrodes simultaneously while they are placed together (e.g., by bundling), but In other examples, any of the aforementioned membrane systems are deposited onto each individual working electrode first, and the two working electrodes are then placed together.

FIG. 2A is a cross-sectional view through the exemplary sensor of FIG. 1A-1C, illustrating one example of sensing membrane 400. In this particular non-limiting example, the membrane system includes an electrode layer 420, an enzyme layer 440, a diffusion resistance layer 460, and a biointerface layer and/or drug releasing layer 480, all of which are located around a working electrode of the sensor 100, and all of which are described in more detail elsewhere herein. In some examples, a unitary diffusion resistance domain and biointerface layer and/or drug releasing layer can be included in the membrane system (e.g., wherein the functionality of both layers is incorporated into one domain). In some examples, the sensor is configured for short-term implantation (e.g., from about 1 to 30 days). However, it is understood that the sensing membrane 400 can be modified and/or used in other devices, for example, by including only one or more of the domains, or including additional domains.

FIG. 2B is a cross-sectional view through one example of the sensor, illustrating another example of the membrane system 400. In this particular example, the membrane system includes an interference reduction or blocking layer 430, an enzyme layer 440, a diffusion resistance layer 460, and a biointerface layer and/or drug releasing layer 480 located around the working electrode of a sensor 100, all of which are described in more detail elsewhere herein.

FIG. 2C is a cross-sectional view through one example of the sensor, illustrating still another example of the sensing membrane 400. In this particular example, the membrane system includes an interferent reduction or blocking layer 430, an enzyme layer 440, and a unitary diffusion resistance/biointerface layer 470 located around the working electrode of a sensor and drug releasing layer 480 (not shown), all of which are described in more detail elsewhere herein.

Sensing membranes 400 of some examples can also include a plurality of domains or layers including, for example, an optional electrode domain (e.g., as illustrated in the FIG. 2A), an interference reduction or blocking domain (e.g., as illustrated in FIGS. 2B and 2C), enzyme domain 108, or a cell disruptive domain (not shown).

### Meniscus Coating Apparatus and Method

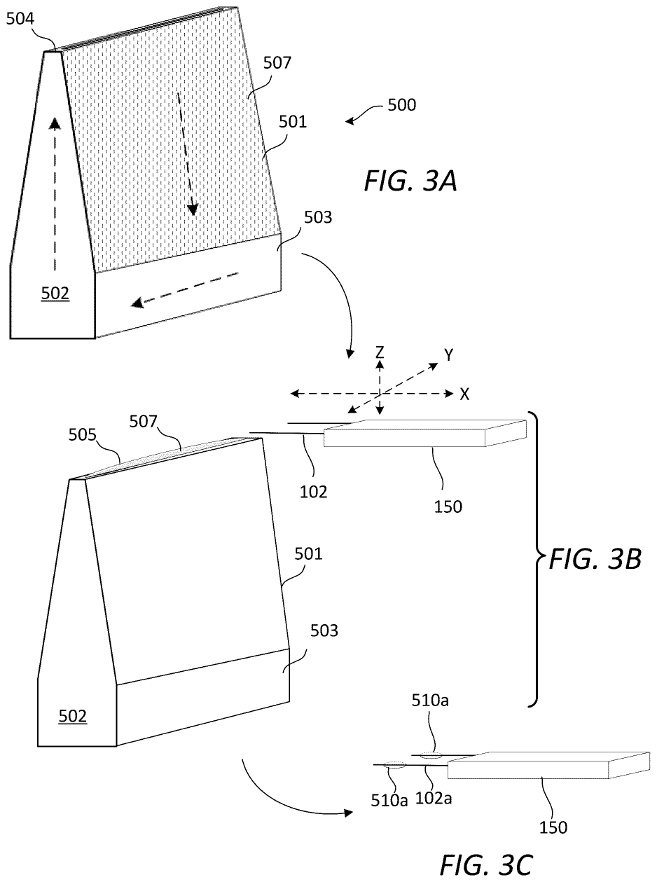

With reference now to FIG. 3A is a perspective view schematic illustrating a meniscus coating apparatus 500, having a base 503 and vertically projecting sides 502 that converge at a distal end forming an opening 504 to provide a meniscus. In examples, the opening 504 has a rectangular shape, a rounded rectangle shape, a stadium shape, a squircle shape, a frustum shape, or a slit shape. In examples, the opening 504 has a square or rounded castellated arrangement about at least a portion of the opening (not shown) with the spacings of the castellated structure larger than the diameter of the elongated body to be presented thereto. In examples, the opening 504 has a width and a length greater than the width. In examples, the width to length ratio is less than 0.1. In examples, the opening 504 is continuous along its length. In examples, the opening 504 is discontinuous along its length.

Coating solution 507 is manipulated vertically through base 503 and opening 504 and allowed to flow down vertical face 501 in a continuous or semi-continuous manner using conventional fluid handling equipment. In examples, the fluid handling equipment includes a reservoir operatively coupled to a pump. The pump, for example, is a low shear pump or a peristaltic pump, or a positive displacement pump that provides stable flow.

In some examples the pump is configured to continuously, semi-continuously or pulsatile present the coating solution 507 to the opening 504. Fluid flow is maintained through a channel entering base 503 which serves as a main fluid line. In examples, three separate siphons are used to transport coating solution from main fluid line to the meniscus 505 at opening 504. In examples, fluid flow is controlled by a pump which circulates the coating solution at a constant rate. In examples, the cross section of the apparatus 500 is generally triangular in shape so as to facilitate a good control on the meniscus height. In examples, a feedback loop is established for viscosity control and can include a viscometer and a solvent injection set up so as to maintain fluid viscosity and/or solid content consistency throughout the coating operation. In examples, a separate stage is connected to the apparatus 500 to allow for holder 150 to be positioned/indexed coating devices.

Vertically projecting sides 502 and face 501 can be substantially perpendicular or angled between 45-89 degrees. Apparatus 500 in examples is configured to heat and/or cool coating solution 507 to a set point or range of temperature. Apparatus 500 in examples is configured to control the flow rate of coating solution 507.

FIGS. 3B, 3C schematically illustrate selectively positioned meniscus coating a plurality of elongated bodies using the meniscus coating apparatus 500, where a continuous or semi-continuous flow of coating solution 507 is stopped so as to form meniscus 505 of coating solution 507 at opening 504. Flow of coating solution 507 can then be restored to refresh the meniscus for additional coatings.

The height of meniscus 505 above the opening 504 can be controlled with a number of process parameters, including, but not limited to, the concentration of material in the coating solution, the solvent used, the temperature of the coating solution, atmospheric pressure, and the viscosity of the coating solution, with the understanding that some parameters are co-dependent and other parameters can be involved and/or manipulated or controlled to provide a desired meniscus height and/or final coating thickness/width along the elongated body.

In examples, one or more process parameters are manipulated or controlled to provide a meniscus height of 0.1-25 mm. The width of opening 504, alone or in combination with the meniscus height can be configured to provide a predetermined coating width and/or thickness on an elongated body 102. The length of opening 504 can be configured to simultaneously coat a plurality of elongated bodies 102.

In examples, holder 150 can be configured to introduce the one or more elongated bodies 102 into the meniscus 505 at a perpendicular angle. In this manner, the meniscus coats a minimal portion of the one or more elongated bodies 102. The minimal portion is determined by the width of the meniscus.

As shown in FIG. 3B, portions of elongated bodies 102 held in holder 150 are introduced to meniscus 505 so as to coat portions of the elongated bodies 102 with coating solution to provide selective coating of coating solution 507 along the longitudinal axis of elongated body 102 as shown in FIG. 3C. Holder 150 and the length of opening 504 can be configured to simultaneously introduce two or more elongated bodies 102 to meniscus 505 for high throughput manufacturing. In examples, holder 150 is computer controlled to translate three-dimensionally along X, Y, Z axes relative to opening 504 of coating apparatus 500 and/or to introduce one or more elongated bodies 102 to meniscus 505 for a predetermined time interval. In examples, holder 150 is configured to piezoelectrically vibrate the elongated bodies 102 so as to modulate the surface tension of the coating solution 507 as it coats, to improve the uniformity of the coating to the elongated body.

In other examples, alone or in combination with the piezoelectric vibration, an electrical impulse is applied to cause the contact angle of the coating solution 507 to change, and e.g., to provide for the coating solution to wet out more like an oil, than a water drop, and/or facilitate wetting and wicking of the coating solution 507.

Elongated body 102 can be pre-treated so as to allow coating solution 507 to disperse or “wet-out” about the circumference surface of elongated body 102. In examples, coated elongated body 102a is dried and/or cured using heat, air flow, light, or combinations thereof, for example to allow solvent of the coating solution 507 to evaporate. It is understood that the height of meniscus 505 and the coating thickness on the elongated body may or may not be the same, for example, due to solvent evaporation rate, and other parameters used for drying or curing. In examples, one or more of the above process parameters are manipulated or controlled to provide a final (dried and/or cured) coating thickness 510a of 0.1-25 mm, 0.5-20 mm, 1-15 mm, or 2-10 mm. In examples, one or more of the above process parameters are manipulated or controlled to provide one or more final (dried and/or cured) coating widths of, independently, 1%, 2%, 3%, 4%, 5%, 6%, 7%, 8%, 9%, 10%, 15%, 20% or more of the elongated body length.

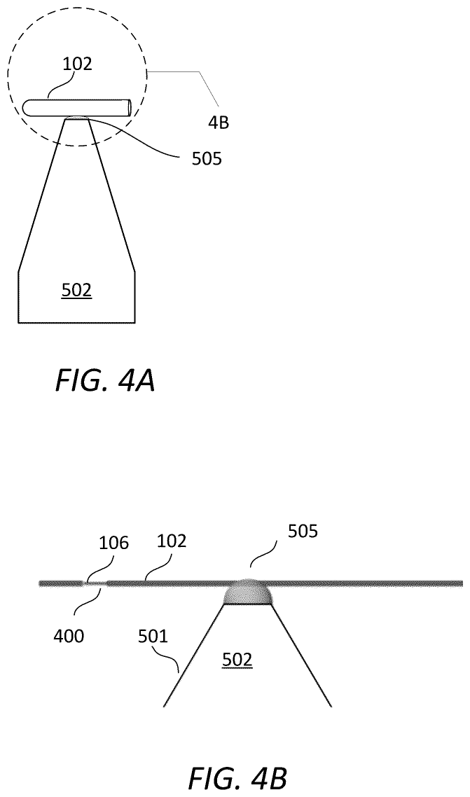

With reference now to FIG. 4A, a side perspective view schematic illustrating an elongated body being selectively positioned for coating using meniscus coating apparatus 500 is depicted. FIG. 4B is an enlarged view of the section 4B of FIG. 4A showing introduction of the elongated body 102 to the meniscus 505 and providing a coating of a layer longitudinally distal from window 106 and sensing membrane therein. Such distal positioning from the window 106 and/or sensing membrane 400, for example, having a drug releasing layer 480, has demonstrated improved performance (e.g., reduction of “lag” compared to a coating location closer to window 106) during the initial implantation of the elongated body of a continuous analyte sensor. It is understood that the coating can be proximal to window 106 on either side thereof and can include the distal tip of elongated body 102. In examples, the coating applied by the apparatus and method described above is longitudinally distal from window 106 on one or both sides of the window. In examples, the coating applied by the apparatus and method described above is absent from window 106. Meniscus coating apparatus 500 is configured to be used alone or in combination with any of the other coating apparatus disclosed herein or with other coating apparatuses and methods.

### Vertical Meniscus/Slit Die Coating Apparatus and Method

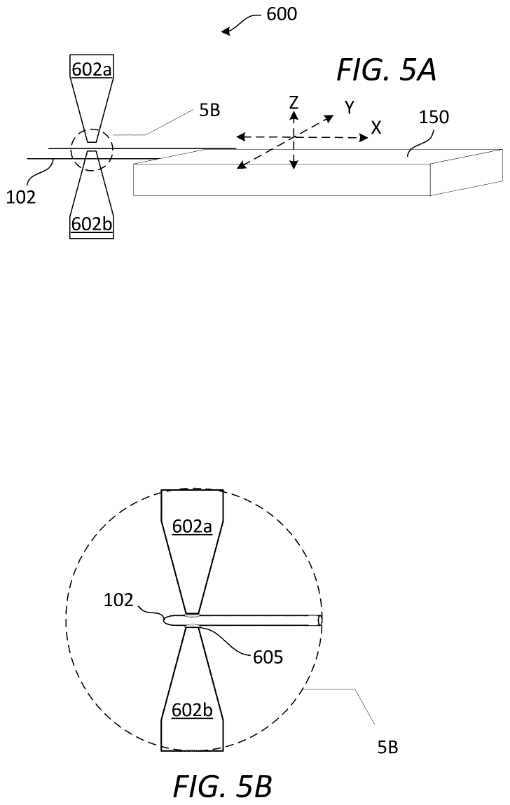

With reference now to FIG. 5A a schematic illustrating selective positional coating to coat one or more elongated bodies using a vertical meniscus coating apparatus 600 comprised of arranged apparatuses 602a, 602b vertically spaced apart a distance that accommodates a diameter of elongated body 102 therebetween. FIG. 5B is an enlarged section of FIG. 5A, and the figures show the coating method of elongated body 102 using holder 150 configured to be computer controlled to translate along axes X, Y, Z relative to opening between apparatuses 602a, 602b and/or to introduce one or more elongated bodies 102 to vertical meniscus 605 (or “curtain”) between apparatuses 602a, 602b for a predetermined time interval. In examples, the apparatus 600 can be configured as a computer controlled slit or die coater configured to introduce coating solution 507 to one or more stationary elongated bodies 102. Meniscus coating apparatus 600 is configured to be used alone or in combination with any of the other coating apparatus disclosed herein or with other coating apparatuses and methods.

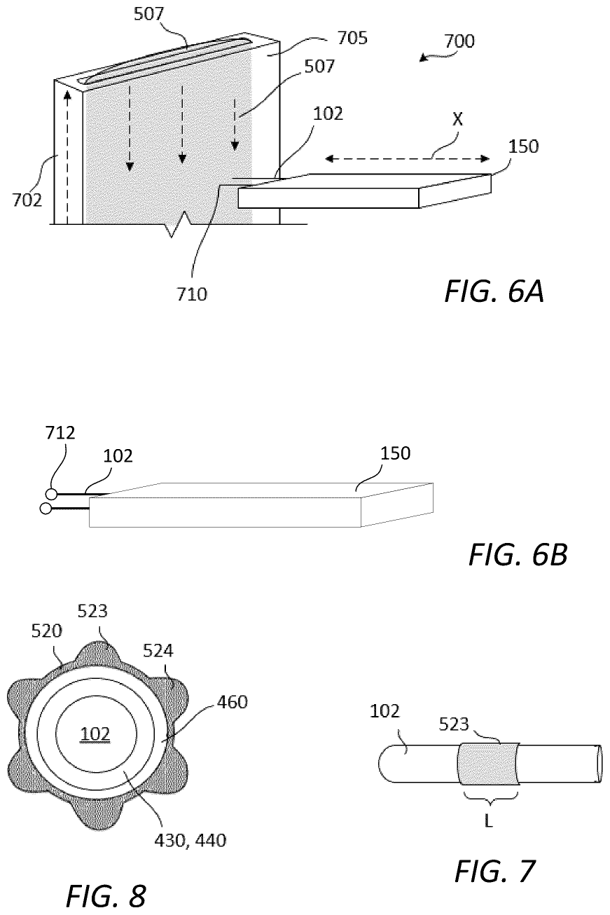

FIGS. 6A and 6B illustrate tip coating of a plurality of elongated bodies using a wall meniscus coating apparatus 700 is shown, where the coating solution 507 is continuously flows upward (arrows 702) and over a vertical face 705 of apparatus 700, the coating solution 507 having a thickness, and distal end 710 of one or more elongated bodies 102 are introduced to the thickness of the coating solution 507 by holder 150 to provide a distal or tip coating 712 on the one or more elongated bodies 102. Holder 150 configured to be computer controlled to translate along at least axes X relative to wall meniscus and thickness of coating solution 507. Elongated bodies 102 are produced with a tip coating 712. In examples, elongated bodies 102 are produced with a tip coating 712 distal from window 106 with sensing membrane 400 (not shown). Meniscus coating apparatus 700 is configured to be used alone or in combination with any of the other coating apparatus disclosed herein or with other coating apparatuses and methods.

In some aspects, the one or more elongated bodies 102 are introduced into coating solution 507 until contact is made with the vertical face 705 of the apparatus 700. In some aspects, the one or more elongated bodies 102 are introduced into the coating solution 507 until contact is made with the vertical face 705 by each tip of the one or more elongated bodies 102. An advantage of contacting each tip is that any differences in the lengths of the elongated bodies 102 extending from holder 150 can be accounted for to ensure that every elongated bodies 102 receives a tip coating 712. Depending on the fixturing, the one or more elongated bodies 102 can slide in the X direction within the holder 150 to align each tip while simultaneously providing the tip coating 712. Alternatively, a portion of the one or more elongated bodies 102 may flex when contacting the vertical face 705 to each receive the tip coating 712 while being securely affixed within the holder 150.

FIGS. 7 and 8, illustrate elongated body 102 with a selectively positioned coating 523, with coating 523 having a length (L) and a thickness. In examples, thickness of coating 523 is essentially a uniform shape about the circumference of elongated body 102. In examples, thickness of coating 523 is non-uniform shape about the circumference of elongated body 102. For example, with reference now to FIG. 8, a sectional view through a longitudinal axis of elongated body 102 illustrates a selectively positioned coating having defined structural contouring about the circumference of elongated body 102. As shown in FIG. 8, the shape of non-uniform coating 523 includes additional surface area in portions 524 in addition to portions 520, which, if comprising a releasable drug, would provide for increased surface area to affect drug elution profile.

In examples, non-uniform coating 523 as shown by example in FIG. 8 is an outer drug-releasing membrane provided by the apparatuses and methods disclosed herein. In examples, a sensor with the outer drug-releasing membrane is configured to be inserted in a subject with a needle-type inserter, where the sensor and outer drug-releasing coating is configured to decrease overall needle-to-drug membrane surface interaction area, thus minimizing needle stiction that otherwise may occur with a uniform coating. In examples, non-uniform coating 523 as shown by example in FIG. 8 is an outer drug-releasing membrane provided by the apparatuses and methods disclosed that helps maximize the total volume of the drug containing membrane and thereby helping maximizing possible effectiveness of the drug. Non-uniform coating 523 as shown by example in FIG. 8 is configured to be used alone or in combination with any of the other coating apparatus disclosed herein or with other coating apparatuses and methods.

### Microfluidic Coating Apparatuses and Methods

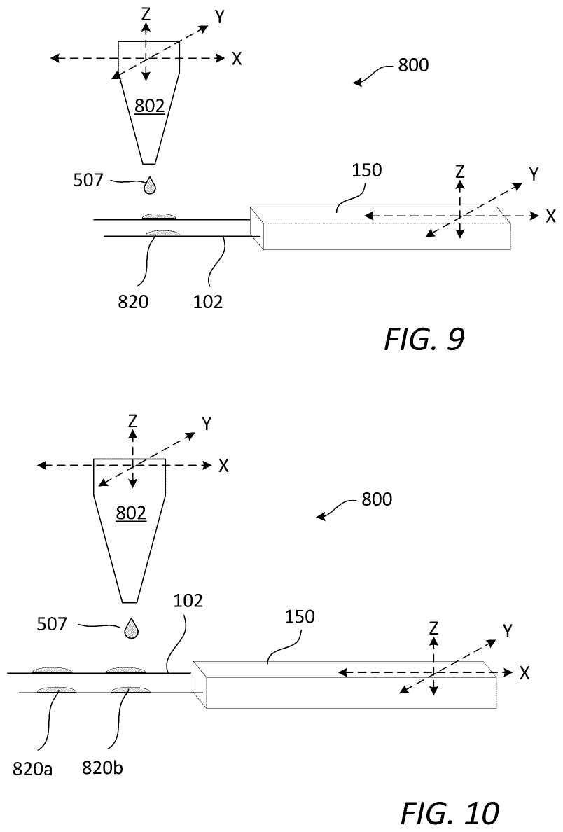

FIG. 9 illustrates a selective positional microfluidic coating apparatus 800 comprising microfluidic coaters 802 configured to introduce a microfluidic amount of coating solution 507 from head 802 along a portion of a longitudinal length of one or more elongated bodies 102. Likewise, as depicted in FIG. 10, selective positional coating heads 820a, 820b of coating solution 507 along a plurality of portions of a longitudinal length of one or more elongated bodies 102 using microfluidic coating apparatus 800 is shown. Holder 150 and heads 820a, 820b, independently, are configured to be computer controlled to translate along axes X, Y, Z to provide one or more coatings, the one or more coatings each having a longitudinal length along the longitudinal axis of the elongated body and thickness (vertically for planar substrates or circumferentially for wire substrates).

In examples, the one or more coating heads 820a, 820b of FIG. 10 are spatially separated along the longitudinal axis of the elongated body. In examples, some or all of the one or more coating heads 820a, 820b meniscus lengths at least partially overlap each other along the longitudinal axis of the elongated body. In examples, the one or more coating heads 820a, 820b comprise the same or different compositions of coating material, concentrations of drug, enzyme, or polymer. In examples, the one or more coating heads 820a, 820b are of different lengths and/or thickness along the longitudinal axis of the elongated body.

Microfluidic coating apparatus 800 is configured to be used alone or in combination with any of the other coating apparatus disclosed herein or with other coating apparatuses. An example of a suitable microfluidic coating apparatus is commercially available PICO® systems sold by Nordson Engineered Fluid Dispensing (EFD) (Westlake, OH).

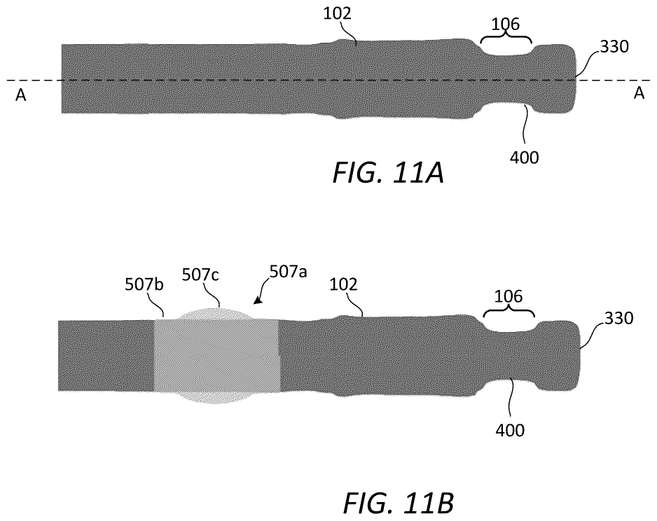

FIG. 11A illustrates an uncoated analyte sensor having an elongated body 102 and FIG. 11B illustrates an analyte sensor having an elongated body 102 body with a portion thereof selectively coated along a longitudinal axis A-A. Coated portion 507a having structural contouring 507b and thickness 507c along the axis A-A. As shown in FIG. 11B, coated portion 507a is distal from window 106 and/or sensing membrane 400. In examples, a sensing membrane is provided by dip-coating processes and coated portion 507a is subsequently provided by one of the coating apparatuses 500, 600, or 800 disclosed herein.

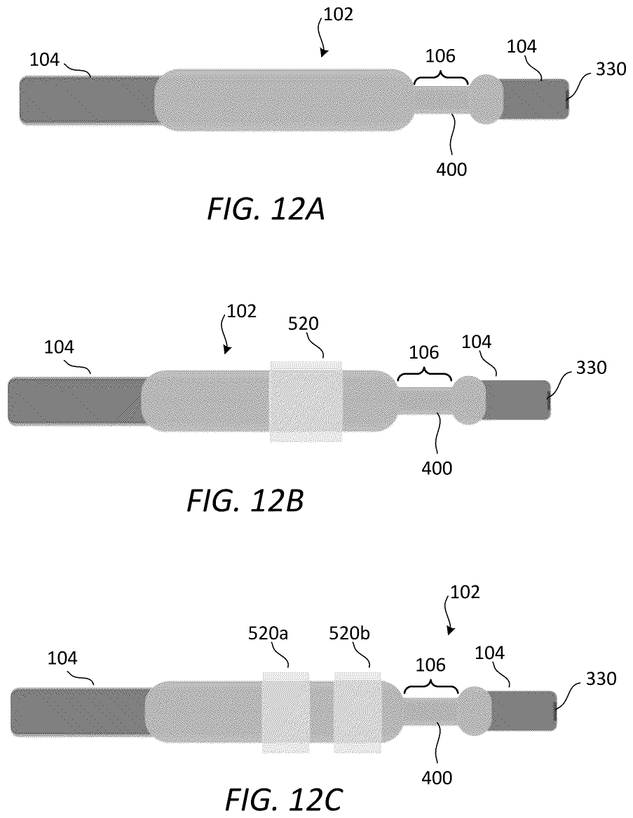

FIG. 12A illustrates an uncoated analyte sensor having elongated body 102 with insulating second layer 104, window 106 exposing an electroactive surface, sensing membrane 400 within window 106, and a distal secondary electrode 330. FIGS. 12B, 12C illustrate the analyte sensor having elongated body 102 of FIG. 12A having a selectively positioned coating portion 520 distal from window 106 and/or sensing membrane 400, or plurality of selectively positioned coating portions 520a, 520b each distal from window 106 and/or sensing membrane 400, respectively.

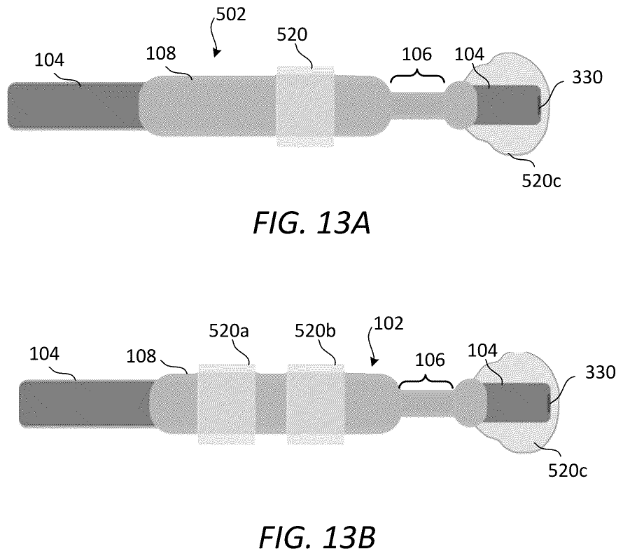

FIGS. 13A and 13B illustrate an analyte sensor having elongated body 102 with a distal secondary electrode 330 having selectively positioned coating 520 or a plurality of selectively positioned coating portions 520a, 520b each distal from window 106 and/or sensing membrane 400, in combination with a distal tip coating 520c as disclosed and described herein. Distal tip coating 520c can be provided by dip coating or coating process 700 described herein.

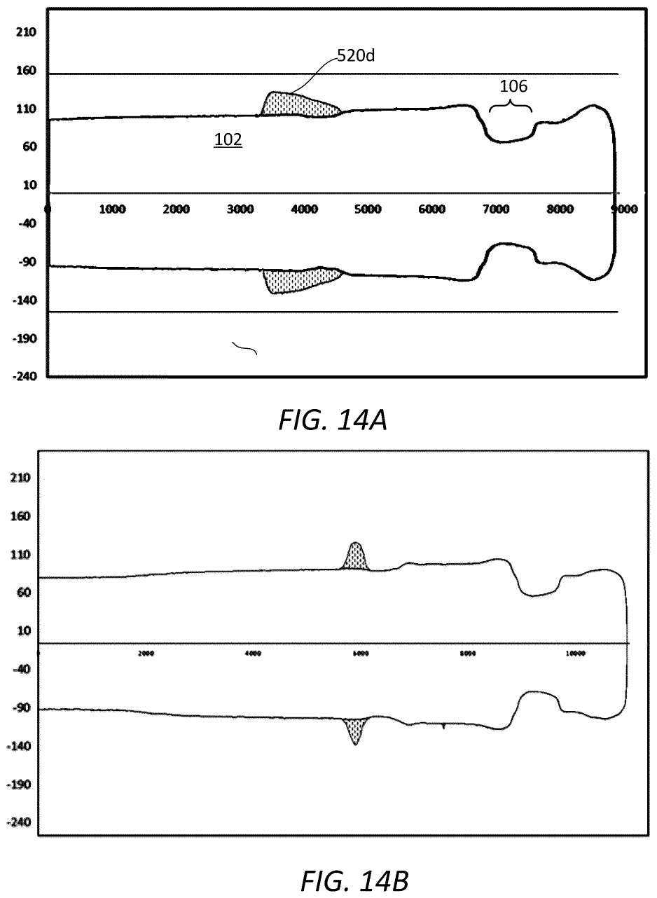

FIGS. 14A and 14B are sectional views along a longitudinal axis of the analyte sensor body illustrating a selectively positioned meniscus coating 520d having defined structural contouring and a selectively positioned microfluidic coating 520e having defined structural contouring, respectively. Combinations of such contouring of selectively positioned coatings along a longitudinal length of an elongated body 102, for example, a wire or planar analyte sensor are envisaged using the coating apparatuses and methods disclosed herein.

### Planar Elongated Body Substrate Coating

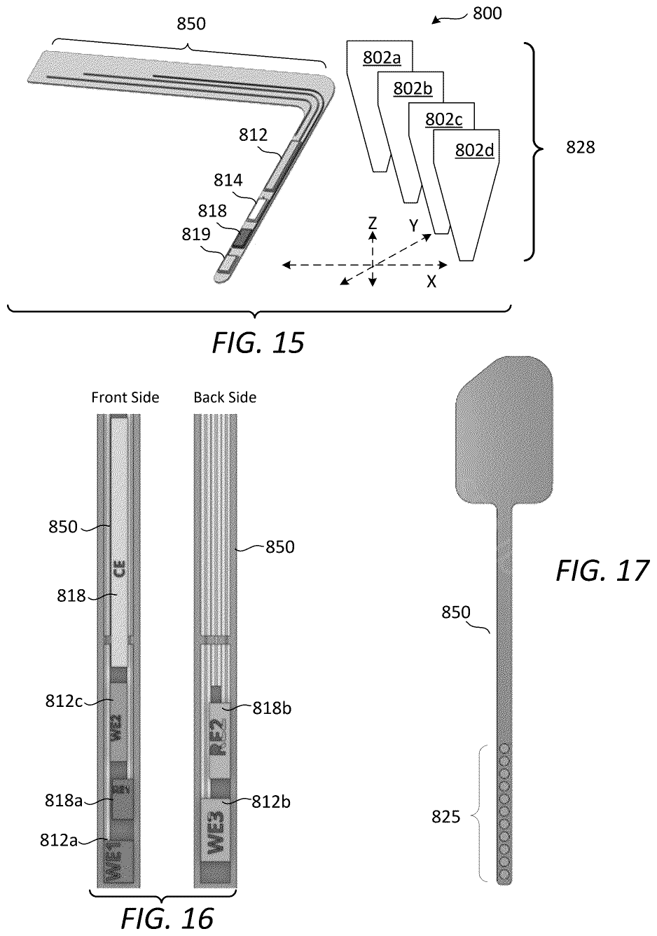

FIGS. 15, 16, and 17, illustrate multiple selective positional coating of a planar analyte sensor body 850 using an array coating apparatus 828 is shown. In examples, array coating apparatus 828 comprises one or more microfluidic coaters. In examples, array coating apparatus 828 comprises a plurality of microfluidic coaters 802a, 802b, 802c, 802d, etc., as shown, each of the plurality of microfluidic coaters being configured to translate along X, Y, and Z axes and to provide coating solution along spatially separated portions 812, 812a, 812b, 812c, 814, 818, 818a, 818b, 819, or 825 etc. of one or both of front/back sides of planar analyte sensor body 850 (FIG. 16, 17). In examples, each of the microfluidic coaters 802a, 802b, 802c, 802d, etc., comprise different coating solutions, or coating solutions with the same compositions but with different concentrations. Other combinations of compositions, concentrations, solvents etc. are envisaged among each of the microfluidic coaters 802a, 802b, 802c, 802d, etc.

In examples, the sensor is an enzyme-based electrochemical sensor, and at least one working electrode measures electronic current or via direct electron transfer of a redox system, e.g., a “wired enzyme” system, and as is appreciated by one skilled in the art. One or more potentiostats is employed to monitor the electrochemical reaction at the electroactive surface of the working electrode(s). The potentiostat applies a constant potential to the working electrode and its associated reference electrode to determine the current produced at the working electrode. The current that is produced at the working electrode (and flows through the circuitry to the counter electrode) is substantially proportional to the amount of H2O2 that diffuses to the working electrode or analyte that facilitates electron transfer in the wired enzyme system. The output signal is typically a raw data stream that is used to provide a useful value of the measured analyte concentration in a host to the host or doctor, for example.

In general, it should be understood that the disclosed coating examples are applicable to a variety of analyte sensor configurations. While some figures herein illustrate sensors that may have a coaxial core and a circular or elliptical cross-section, in other examples of sensor systems including biointerface/drug release layer(s), the sensor may be a substantially planar sensor having a rectangular cross-section.

### Coating Solutions

In examples, the presently disclosed coating solution used in the apparatuses and methods described herein comprises at least one polymer and at least one anti-inflammatory or tissue modifying agent. It is to be understood that coating solutions herein are inclusive of any or all of the components of sensing membrane 400 and other components of a continuous analyte monitoring device. For example, in some examples, the coating solution comprises one of sensing membrane 400 components, e.g., one electrode layer, one enzyme layer, and one or more biointerface layers. In other examples, the coating solution used in the apparatuses and methods described herein comprises one or more polymers and one or more anti-inflammatory agents or tissue modifiers.

In some examples, the coating solution 507 or the sensing membrane 400 comprises enzymes or enzyme solutions, alone or in combination with any of the above materials. For example, the coating solution 507 can comprise one or more enzymes selected from glucose oxidase, glucose dehydrogenase, β-hydroxybutyrate dehydrogenase, or lactate dehydrogenase. In other examples, the coating solution 507 can comprise other oxidases, including, for example, galactose oxidase, cholesterol oxidase, an amino acid oxidase, alcohol oxidase, lactate oxidase, aspartate oxidase, or uricase. In examples, the sensing membrane 400 can include one or more of glucose dehydrogenase, lactate dehydrogenase, malate dehydrogenase, glycerol dehydrogenase, alcohol dehydrogenase, sorbitol dehydrogenase, and an amino acid dehydrogenase comprising L-amino acid dehydrogenase, asparaginase, or superoxide dismutase.

In examples, the sensing membrane 400 or the one or more coating solutions 507 used in the apparatuses and methods disclosed herein can comprise β-hydroxybutyrate dehydrogenase (HBD) enzyme, a nicotinamide adenine dinucleotide (NAD+) and a metal or non-metal mediator and optionally a crosslinking agent. In examples, the coating solution 507 comprises β-hydroxybutyrate dehydrogenase (HBD) enzyme, a nicotinamide adenine dinucleotide (NAD+) and a metal or non-metal mediator in combination with glucose oxidase or glucose dehydrogenase.

The apparatuses and methods described herein provide for the selective coating of the above sensing membrane components. It is understood that the apparatuses and methods described herein can be combined with the selective coating of sensing membrane components and the selective coating of one or more polymers and one or more anti-inflammatory agents or tissue modifiers.

PCT Publication No. WO2024144921 and U.S. Publication No. 20240090802, which are incorporated herein by reference in their entireties, describe drug releasing-, biointerface-, and sensing-membrane configurations and materials that are to be applied to elongated bodies using the methods and apparatuses as presently disclosed.

It is also understood that the apparatuses and methods described herein can be used to selectively positional coat one or more drug releasing membranes having one or more drugs dispersed, dissolved, or distributed therein using the coating solution 507. Thus, the coating solution can comprise, among other things, anti-inflammatoires, vasodilators, enzymes, co-factors, mediators, polymers, crosslinking agents, and solvents.

In some examples, one or more coating solutions 507 to be coated on a device using the methods and apparatuses disclosed herein comprise one or more materials such as silicone, polytetrafluoroethylene, polyethylene-co-tetrafluoroethylene, polyolefin, polyester, polycarbonate, biostable polytetrafluoroethylene, homopolymers, copolymers, terpolymers of polyurethanes, polyureas, polyurethane ureas, polyethers, polypropylene (PP), polyvinylchloride (PVC), polyvinylidene fluoride (PVDF), polybutylene terephthalate (PBT), polymethylmethacrylate (PMMA), polyether ether ketone (PEEK), cellulosic polymers, poly(ethylene oxide), poly(propylene oxide) and copolymers and blends thereof, heterocyclic nitrogen containing polymers, such as polymers or copolymers of polyvinylpyridine (PVP) and polyvinylimidazole, polysulfones and block copolymers thereof including, for example, di-block, tri-block, alternating, random and graft copolymers, polystyrene copolymers, including polystyrene-polypyridine copolymers and quaternized forms, acrylonitrile, and acrylamide, with or without one or more drugs or active agents. In examples, coating solution 507 comprises a conductive polymer or a doped conductive polymer.

In examples, coating solution 507 comprises one or more anti-inflammatory compounds or tissue response modifiers. In examples, coating solution 507 comprises at least one polymer or polymerizable monomer in combination with at least one anti-inflammatory compounds or tissue response modifiers. In examples, coating solution 507 comprises dexamethasone, dexamethasone acetate, a derivative form thereof, or a combination thereof.

In examples, the coating solution 507 comprises at least one polymer or polymerizable monomer in combination with at least one anti-inflammatory compounds or tissue response modifiers. In examples, the coating solution 507 comprises a polymer chain having polyurethane and/or polyurea segments. In examples, the coating solution 507 comprises a polymer chain having both hydrophilic and hydrophobic regions. In examples, the coating solution 507 comprises a polymer chain having one or more zwitterionic compounds.

### Photon Activated Drug Release

In aspects, the devices and methods disclosed herein are configured for providing stimuli-responsive drug delivery systems (s-DDS). Such systems may overcome one or more drawbacks of sustained release of drugs in interstitial fluid (ISF) compartments surrounding implantable sensors, for example, initial lag time of signal or accurate readings post-implantation. In examples, light-responsive polymer-based s-DDS systems provide spatial and temporal control of drug delivery in interstitial fluid (ISF) compartments surrounding implantable sensors In this regard, near infrared (NIR) light triggered drug nanocarriers present several advantages when compared to UV-visible light triggered nanocarriers.

UV and visible light exhibits low tissue penetration; and can be harmful to cells and living tissue. Because of such disadvantages, UV light responsive materials are difficult to use in clinical situations and with implanted devices. However, near infrared light (˜1100 nm) minimizes scattering and absorption, contributing to much deeper penetration in biological living tissues than UV and visible light. In addition, NIR does not damage tissue or cells to the extent UV does. It is understood that UV and visible light can be combined and/or filtered together with NIR in the present disclosure provided the NIR intensity is greater than either UV or visible light intensity.

In aspects, a photon-activated layer is provided, the photon-activated layer providing for direct or indirect photon stimuli-responsive drug release. In examples, the photon-activated layer comprises one or more compounds responsive to photon stimulation.

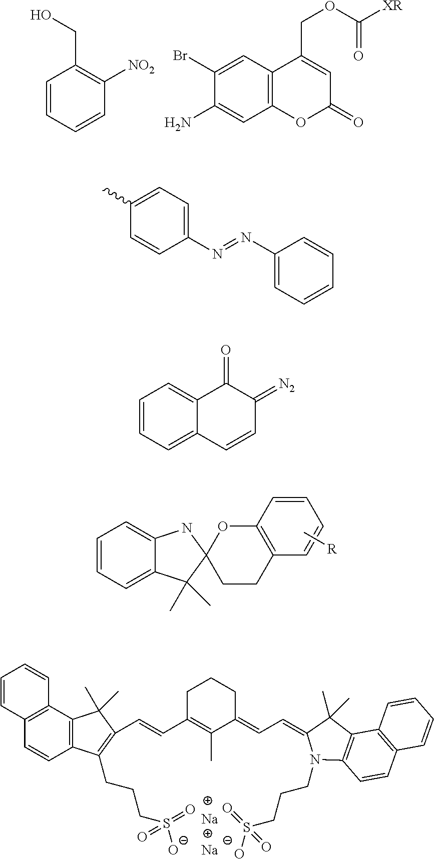

Photo stimulation includes bond scission, molecular degradation, and/or free radical or ionic species generation. In examples, bond scission, molecular degradation, and/or free radical or ionic species generation provides for new or additional diffusion pathways of drug through the photon-activated layer, thus, increasing the amount and or rate of drug released. In examples, the photon-activated layer comprises one or more anti-inflammatory or tissue response modifying drugs. Examples of one or more compounds responsive to photon stimulation are shown in Scheme 1 and include ortho-nitrobenzyl alcohol, coumarin derivatives, where X is nitrogen, oxygen or sulfur and R is alkyl, aryl or substituted forms thereof, diazonaphthoquinone (DNQ), spiropyran, trans-azobenzene derivative, and indocyanine green (ICG).

To avoid the damage of living tissue and the low penetration of UV/vis light, one alternative way is the use of two-photon absorption (2 PA) compounds. These latter, through the absorption of two photons simultaneously, are capable of converting the excitation wavelengths from the visible to the NIR region.

In other examples, the photon-activated layer is an adjacent a drug release layer that comprises one or more anti-inflammatory or tissue response modifying drugs and is positioned below the photon-activated layer and prevents or reduces drug release from the drug releasing layer prior to exposure to photons, and upon exposure to photons, provides for an increase of the amount or the rate of release of the one or more comprises one or more anti-inflammatory or tissue response modifying drugs.

In examples, photon-activated layer is activated by UV light, visible light, near IR light, far IR light or combinations thereof. In examples, photon-activated layer is activated by near IR light (NIR), which can penetrate skin and is less damaging than UV light. In examples, at least a portion of a sensor housing affixed to the skin of a user and positioned above the implanted sensor is configured for NIR light transmission to facilitate photon activation of the photon-activated layer. In examples, NIR light is provided by one or more NIR light sources within the sensor housing.

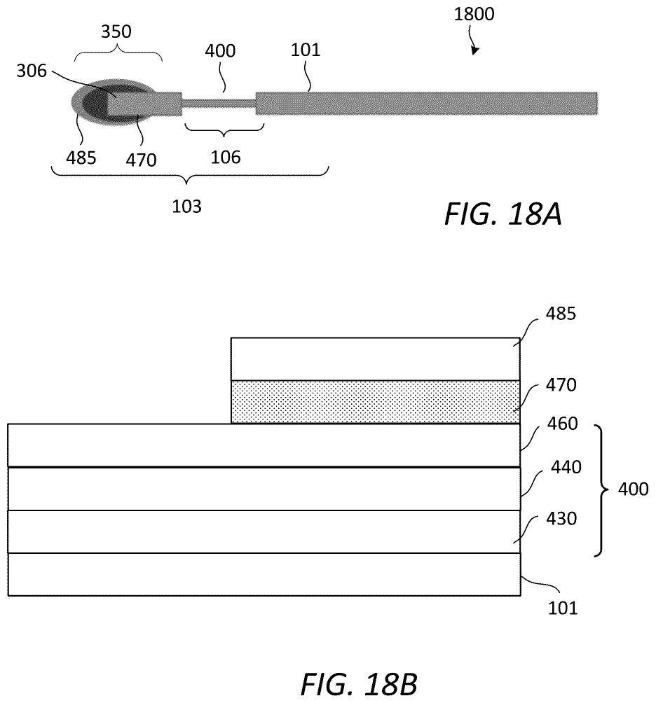

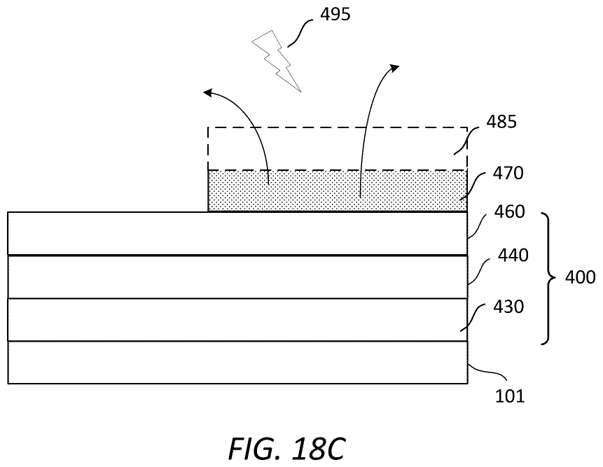

With reference to FIG. 18A, an elongated body sensor 1800 is shown with distal tip 306 coated with membrane stack 350 comprising drug releasing layer 470 and photon-activated layer 485. As shown, drug releasing layer 470 and photon-activated layer 485 are distal from window 106 and sensing membrane 400 of implantable portion 103. Drug releasing layer 470 and photon-activated layer 485 are provided using the coating methods and devices disclosed herein or by dip coating techniques.

An exemplary photon-activated layer membrane stack is shown in FIG. 18B. comprising the anti-inflammatory or tissue response modifying drug layer 470 shown adjacent sensing membrane 400 which can include blocking layer 430, enzyme layer 440, and diffusion resistance layer 460 provided on a substrate 101 which can be of a wire or planar construction. Photon activated layer 485 can be positioned adjacent or directly adjacent at least part of drug layer 470 to modulate, adjust, assist, or control drug release.

As shown in FIG. 18C, upon exposure to photons, photon-activated layer 485 and allows components of underlying drug releasing layer 470 to release drug, as shown by arrows. In examples, photon-activated layer 485 is configured, after exposure to photons 495, to at least partially degrade, provide one or more channels capable of assisting diffusion of drug from drug layer 470, or cause accelerated biological degradation of photon-activated layer 485. The amount or intensity of the photons can be configured to provide sufficient activation of the photon-sensitive compound.

Permeability of the photon-activated layer is adjusted by chemical property of the polymer matrix, amount of photon-sensitive compound. In examples, compounds and wavelengths of the photon are chosen to provide for two-photon excitation (2 PE) processes in the photon-activated layer 485. Two photon absorption (2 PA) engaging the two simultaneously absorbed photons of identical or different frequencies to excite a molecule from its ground state to a higher energy state. The use of light sensitive compounds exhibiting two-photon excitation (2 PE) process can improve cell survival and tissue penetration relative to conventional one-photon excitation (1 PE) compounds (UV sensitive compounds).

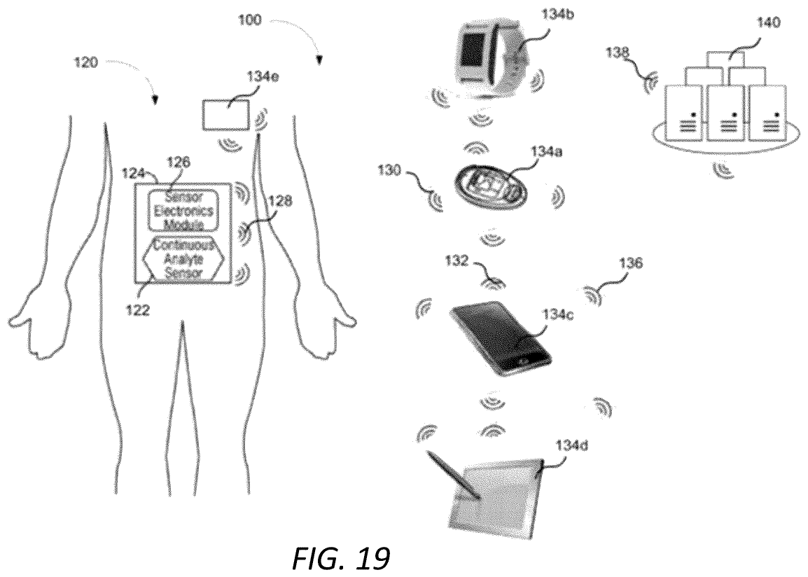

FIG. 19 is a diagram depicting an example continuous transcutaneous analyte monitoring system 100 configured to measure one or more analytes and/or electrophysiological indicators (e.g., blood pressure, heart rate, core temperature, etc.). The monitoring system includes a continuous transcutaneous analyte sensor system 124 operatively connected to a host 120 and a plurality of display devices 134 a-e according to certain aspects of the present disclosure. It should be noted that display device 134e alternatively or in addition to being a display device, may be a medicament delivery device that can act cooperatively with the continuous transcutaneous analyte sensor system 124 to deliver medicaments to host 120. The continuous transcutaneous analyte sensor system 124 may include a sensor electronics module 126 and a continuous transcutaneous analyte sensor 122 associated with the sensor electronics module 126. The sensor electronics module 126 may be in direct wireless communication with one or more of the plurality of the display devices 134a-e via wireless communications signals. In one example, display devices 134a-e may also communicate amongst each other and/or through each other to continuous transcutaneous analyte sensor system 124. For ease of reference, wireless communications signals from analyte sensor system 124 to display devices 134a-e can be referred to as “uplink” signals 128. Wireless communications signals from, e.g., display devices 134a-e to continuous transcutaneous analyte sensor system 124 can be referred to as “downlink” signals 130. Wireless communication signals between two or more of display devices 134a-e may be referred to as “crosslink” signals 132. Additionally, wireless communication signals can include data transmitted by one or more of display devices 134a-d via “long-range” uplink signals 136 (e.g., cellular signals) to one or more remote servers 140 or network entities, such as cloud-based servers or databases, and receive long-range downlink signals 138 transmitted by remote servers 140.

The sensor electronics module 126 includes sensor electronics that are configured to process sensor information and generate transformed sensor information. In certain examples, the sensor electronics module 126 includes electronic circuitry associated with measuring and processing data from continuous transcutaneous analyte sensor 122, including prospective algorithms associated with processing and calibration of the continuous transcutaneous analyte sensor data. The sensor electronics module 126 can be integral with (non-releasably attached to) or releasably attachable to the continuous transcutaneous analyte sensor 122 achieving a physical connection therebetween. The sensor electronics module 126 may include hardware, firmware, and/or software that enables analyte level measurement. For example, the sensor electronics module 126 can include a potentiostat, a power source for providing power to continuous transcutaneous analyte sensor 122, other components useful for signal processing and data storage, and a telemetry module for transmitting data from itself to one or more display devices 134a-e. Electronics can be affixed to a printed circuit board (PCB), or the like, and can take a variety of forms. For example, the electronics can take the form of an integrated circuit (IC), such as an Application-Specific Integrated Circuit (ASIC), a microcontroller, and/or a processor. Examples of systems and methods for processing sensor analyte data are described in more detail herein and in U.S. Pat. Nos. 7,310,544 and 6,931,327 and U.S. Patent Publication Nos. 2005/0043598, 2007/0032706, 2007/0016381, 2008/0033254, 2005/0203360, 2005/0154271, 2005/0192557, 2006/0222566, 2007/0203966 and 2007/0208245, each of which are incorporated herein by reference in their entirety for all purposes.

Display devices 134a-e are configured for displaying, alarming, and/or basing medicament delivery on the sensor information that has been transmitted by the sensor electronics module 126 (e.g., in a customized data package that is transmitted to one or more of display devices 134a-e based on their respective preferences). Each of the display devices 134a-e can include a display such as a touchscreen display for displaying sensor information to a user (most often host 120 or a caretaker/medical professional) and/or receiving inputs from the user. In some examples, the display devices 134a-e may include other types of user interfaces such as a voice user interface instead of or in addition to a touchscreen display for communicating sensor information to the user of the display device 134a-e and/or receiving user inputs. In some examples, one, some or all of the display devices 134a-e are configured to display or otherwise communicate the sensor information as it is communicated from the sensor electronics module 126 (e.g., in a data package that is transmitted to respective display devices 134a-e), without any additional prospective processing required for calibration and real-time display of the sensor information.

In the example of FIG. 19, one of the plurality of display devices 134a-e may be a custom display device 134a specially designed for displaying certain types of displayable sensor information associated with analyte values received from the sensor electronics module 126 (e.g., a numerical value and an arrow, in some examples). In some examples, one of the plurality of display devices 134a-e may be a handheld device 134c, such as a mobile phone based on the Android, IOS operating system or other operating system, a palm-top computer and the like, where handheld device 134c may have a relatively larger display and be configured to display a graphical representation of the continuous sensor data (e.g., including current and historic data). Other display devices can include other hand-held devices, such as a tablet 134d, a smart watch 134b, a medicament delivery device 134e, a blood glucose meter, and/or a desktop or laptop computers.

As alluded to above, because the different display devices 134a-e provide different user interfaces, content of the data packages (e.g., amount, format, and/or type of data to be displayed, alarms, and the like) can be customized (e.g., programmed differently by the manufacture and/or by an end user) for each particular display device and/or display device type. Accordingly, in the example of FIG. 19, one or more of display devices 134a-e can be in direct or indirect wireless communication with the sensor electronics module 126 to enable a plurality of different types and/or levels of display and/or functionality associated with the sensor information, which is described in more detail elsewhere herein.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

All numbers expressing quantities of ingredients, reaction conditions, and so forth used in the specification are to be understood as being modified in all instances by the term “about.” Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

The term “comprising” as used herein is synonymous with “including,” “containing,” or “characterized by,” and is inclusive or open-ended and does not exclude additional, unrecited elements or method steps.

The above description discloses several methods and materials of the present disclosure. This disclosure is susceptible to modifications in the methods and materials, as well as alterations in the fabrication methods and equipment. Such modifications will become apparent to those skilled in the art from a consideration of this disclosure or practice of the disclosure disclosed herein. Consequently, it is not intended that this disclosure be limited to the specific examples disclosed herein, but that it covers all modifications and alternatives coming within the true scope and spirit of the disclosure. While certain examples of the present disclosure have been illustrated with reference to specific combinations of elements, various other combinations may also be provided without departing from the teachings of the present disclosure. Thus, the present disclosure should not be construed as being limited to the particular exemplary examples described herein and illustrated in the Figures, but may also encompass combinations of elements of the various illustrated examples and aspects thereof.

1. A device for measurement of an analyte concentration, the device comprising:
an analyte sensing portion configured to generate a signal associated with a concentration of an analyte; and
a bioactive agent releasing portion configured to release a bioactive agent;
wherein the bioactive agent-releasing portion and the analyte sensing portion are spatially separated along a longitudinal axis of the wire substrate or the planar substrate; or
wherein the bioactive agent-releasing portion and the analyte sensing portion are present on separate wire substrates or separate planar substrates.

2. The device of claim 1, wherein the analyte sensing portion is present on the planar substrate.

3. The device of claim 1, wherein the analyte sensing portion is present on the wire substrate.

4. The device of claim 1, wherein the bioactive agent releasing portion is present on the planar substrate.

5. The device of claim 1, wherein the bioactive agent releasing portion is present on the wire substrate.

6. The device of claim 1, wherein the bioactive agent-releasing portion and the analyte sensing portion are spatially separated.

7. The device of claim 1, wherein the bioactive agent-releasing portion and the analyte sensing portion are spatially separated along a longitudinal axis of the wire substrate or the planar substrate.

8. The device of claim 1, wherein the analyte sensing portion comprises a WE, a RE, and/or CE configured to generate a signal associated with the analyte.

9. The device of claim 1, wherein the bioactive agent-releasing portion comprises at least one bioactive agent-releasing electrode.

10. The device of claim 1, wherein the bioactive agent-releasing electrode is distal from the WE or RE.

11. The device of claim 1, wherein the WE and the bioactive agent-releasing electrode share the CE or the RE.

12. The device of claim 1, wherein the analyte sensing portion comprises a first WE configured to generate a signal associated with a first analyte.

13. The device of claim 1, wherein the analyte sensing portion comprises a second WE configured to generate a signal associated with a second analyte, the second analyte being chemically different from the first analyte.

14. The device of claim 1, wherein the bioactive agent-releasing electrode is positioned most distal relative to any other WE electrode.

15. The device of claim 1, further comprising an electrically conductive membrane in proximity to the bioactive agent-releasing electrode, the electrically conductive membrane comprising at least one bioactive agent, the at least one bioactive agent configured to be released from the electrically conductive membrane to modify tissue response of a subject.

16. The device of claim 15, wherein the electrically conductive membrane comprises at least one electrically conductive polymer.

17. The device of claim 16, wherein the at least one electrically conductive polymer is doped.

18. The device of claim 1, wherein the signal is potentiometric, coulometric, or amperometric.

19. A device for measurement of an analyte concentration, the device comprising:
an analyte sensing portion configured to generate a signal associated with a concentration of an analyte;
a bioactive agent releasing portion adjacent the analyte sensing portion, the bioactive agent releasing portion configured to release a bioactive agent upon implantation in interstitial fluid; and
a photon-activated layer adjacent the bioactive agent releasing portion, wherein the photon-activated layer comprises one or photo-sensitive agents.

20. A method of controlling drug release from an implantable portion an analyte sensor, the method comprising:
providing an analyte sensor comprising an implantable portion configured to generate a signal associated with a concentration of an analyte;
a bioactive agent releasing portion adjacent the analyte sensing portion, the bioactive agent releasing portion configured to release a bioactive agent upon implantation in interstitial fluid; and
a photon-activated layer adjacent the bioactive agent releasing portion;
wherein the photon-activated layer comprises one or more photo-sensitive agents and reduces a release rate or amount of the bioactive agent;
wherein the photon-activated layer is configured to modify the release rate or the amount of the bioactive agent from the bioactive agent releasing portion upon exposure to an amount of photon energy.
